(19) United States
(12) Patent Application Publication
(10) Pub. No.: US 2005/0222163 A1
(43) Pub. Date: Oct. 6, 2005

(54) Combinations of signal transduction inhibitors

(75) Inventors: Stephen Louis Eck, Ann Arbor, MI (US); David William Fry, Ypsilanti, MI (US); Judith Ann Leopold, Ann Arbor, MI (US)

Correspondence Address: AGOURON PHARMACEUTICALS, INC., 10777 SCIENCE CENTER DRIVE, SAN DIEGO, CA, 92121, US

(73) Assignee: PFIZER INC

(21) Appl. No.: 11/095,442

(22) Filed: Mar. 30, 2005

Related U.S. Application Data
(60) Provisional application No. 60/557,623, filed on Mar. 30, 2004.

(51) Int. Cl. A61K 31/519

(52) U.S. Cl. 514/252.16

(57) ABSTRACT

The present invention relates to methods for treating cancer comprising utilizing a combination of signal transduction inhibitors. More specifically, the present invention relates to combinations of so called cell cycle inhibitors with mitogen stimulated kinase signal transduction inhibitors, more specifically combinations of CDK inhibitors with mitogen stimulated kinase signal transduction inhibitors, more preferably MEK inhibitors. Other embodiments of the invention relate to additional combinations of the aforesaid combinations with standard anti-cancer agents such as cytotoxic agents, palliatives and antiangiogenics. Most specifically this invention relates to combinations of 6-acetyl-8-cyclopentyl-5-methyl-2-(5-piperazin-1-yl-pyridin-2-ylamino)-8H-pyrido[2,3-d]pyrimidin-7-one including salt forms, which is a selective cyclin-dependent kinase 4 (CDK4) inhibitor, in combination with one or more MEK inhibitors, most preferably N-[(R)-2,3-dihydroxy-propoxy]-3,4-difluoro-2-(2-fluoro-4-iodo-phenylamino)-benzamide. The aforementioned combinations are useful for treating inflammation and cell proliferative diseases such as cancer and restenosis.

This application claims the benefit of priority from U.S. Provisional Application No. 60/557,623 filed on Mar. 30, 2004.

### BACKGROUND OF THE INVENTION

The present invention relates to methods for treating cancer comprising utilizing a combination of signal transduction inhibitors. More specifically, the present invention relates to combinations of so called cell cycle inhibitors with mitogen stimulated kinase signal transduction pathway inhibitors, more specifically combinations of CDK inhibitors with mitogen stimulated kinase signal transduction inhibitors, more preferably MEK inhibitors. Other embodiments of the invention relate to additional combinations of the aforesaid combinations with standard anti-cancer agents such as cytotoxic agents, palliatives and antiangiogenics. Most specifically this invention relates to combinations of 6-acetyl-8-cyclopentyl-5-methyl-2-(5-piperazin-1-yl-pyridin-2-ylamino)-8H-pyrido[2,3-d]pyrimidin-7-one, which is a selective cyclin-dependent kinase 4 (CDK4) inhibitor in combination with MEK inhibitors, most preferably N-[(R)-2,3-dihydroxy-propoxy]-3,4-difluoro-2-(2-fluoro-4-iodo-phenylamino)-benzamide. The present invention includes all salts, metabolites, prodrugs, isomers and polymorphs of each of the aforementioned active agents. The aforementioned combinations are useful for treating inflammation and cell proliferative diseases such as cancer and restenosis.

Cell cycle inhibitors are key gatekeepers of signal transduction kinases that control the progression of the cell cycle. Cell cycle inhibitors include cyclin dependent kinase inhibitors (CDK), Aurora Kinase inhibitors, PLK inhibitors and CHK1 inhibitors. The preferred cell cycle inhibitors of the present invention are cyclin dependent kinases inhibitors.

Cyclin-dependent kinases and related serine/threonine protein kinases are important cellular enzymes that perform essential functions in regulating cell division and proliferation. The cyclin-dependent kinase catalytic units are activated by regulatory subunits known as cyclins. At least sixteen mammalian cyclins have been identified (D. G. Johnson and C. L. Walker, Annu. Rev. Pharmacol. Toxicol. (1999) 39:295-312). Cyclin B/CDK1, Cyclin A/CDK2, Cyclin E/CDK2, Cyclin D/CDK4, Cyclin D/CDK6, and probably other heterodimers including CDK3 and CDK7 are important regulators of cell cycle progression. Additional functions of Cyclin/CDK heterodimers include regulation of transcription, DNA repair, differentiation and apoptosis (D. O. Morgan, Annu. Rev. Cell. Dev. Biol. (1997) 13261-13291).

Cyclin-dependent kinase inhibitors have been demonstrated to be useful in treating cancer. Increased activity or temporally abnormal activation of cyclin-dependent kinases has been shown to result in the development of human tumors (C. J. Sherr, Science (1996) 274:1672-1677); M. Malumbres, Nature Rev. Cancer, 2001, 1, 222-231 and C. J. Sherr, Cancer Cell, 2002, 2, 103-112. Indeed, human tumor development is commonly associated with alterations in either the CDK proteins themselves or their regulators (C. Cordon-Cardo, Am. J. Pathol. (1995) 147:545-560; J. E. Karp and S. Broder, Nat. Med. (1995) 1:309-320; M. Hall et al., Adv. Cancer Res. (1996) 68:67-108). Naturally occurring protein inhibitors of CDKs such as p16 and p27 cause in vitro growth inhibition in lung cancer cell lines (A. Kamb, Curr. Top. Microbiol. Immunol. (1998) 227:139-148). Certain CDK inhibitors have also been shown to be useful as chemoprotective agents through their ability to inhibit cell cycle progression of normal untransformed cells (Chen et al. J. Natl. Cancer Institute (2000) 92:1999-2008). Several CDK inhibitors are currently under evaluation by pharmaceutical companies although none have yet been approved for commercial use (P. M. Fischer, Curr. Opin. Drug Discovery (2001) 4:623-634; D. W. Fry and M. D. Garrett, Curr. Opin. Oncologic, Endocrine & Metabolic Invest. (2000) 2:40-59; K. R. Webster and D. Kimball, Emerging Drugs (2000) 5:45-59; T. M. Sielecki et al., J. Med. Chem. (2000) 43:1-18; P. Fisher, Curr. Opin. Drug Disc. & Development 2001, 4, 623-634; K. Webster, Emerging Drugs 2000, 5, 45-59; and D. Fry, Curr. Opin. Oncologic, Endocrine & Metabolic Invest. Drugs. 2000, 2, 40-59.

Selective CDK4/6 inhibitors have been disclosed in commonly assigned International Patent Application PCT/IB03/00059, filed Jan. 10, 2003 (the '059 application), which is herein incorporated by reference in its entirety for all purposes. The '059 application discloses a particularly potent and selective CDK4/6 inhibitor, 6-acetyl-8-cyclopentyl-5-methyl-2-(5-piperazin-1-yl-pyridin-2-ylamino)-8H-pyrido[2,3-d]pyrimidin-7-one:

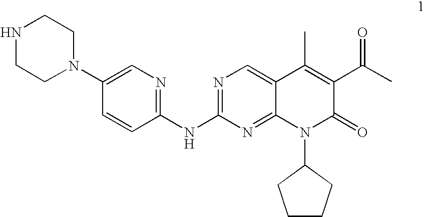

6-acetyl-8-cyclopentyl-5-methyl-2-(5-piperazin-1-yl-pyridin-2-ylamino)-8H-pyrido[2,3-d]pyrimidin-7-one is preferably administered as an isethionate salt. One preferred isethionate salt comprises a mono-isethionate salt of Form A. Another preferred isethionate salt is the salt of polymorphic Form B. Another preferred isethionate sale is the polymorphic salt of Form D.

In standard enzyme assays the compound of Formula 1 exhibits IC₅₀ concentrations for CDK4 and CDK2 inhibition (at 25° C.) of 0.011 μM and >5 μM, respectively. For a discussion of standard CDK4 and CDK2 assays for IC₅₀ determinations, see D. W. Fry et al., J. Biol. Chem. (2001) 16617-16623.

The present invention is primarily directed to combinations of cell cycle inhibitors with other signal transduction inhibitors (STI). STI as used herein refers to inhibitors that produce a direct effect on the signaling pathways that promote growth, proliferation and survival of a cell. Such inhibitors include small molecules, antibodies, and antisense molecules. Examples of such signal transduction inhibitors include tyrosine kinase inhibitors and serine/threonine kinase inhibitors. Such inhibitors include MEK inhibitors, bcr-abl tyrosine kinase inhibitors, PDGFR inhibitors, c-Kit inhibitors, erbB inhibitors, VEGF-R inhibitors, Hsp 90 inhibitors, FLT-3 inhibitors, K-Ras inhibitors, PI3 kinase inhibitor, Raf kinase inhibitors, Akt inhibitors, mTOR inhibitor and multi-targeted kinase inhibitors see “Signal Transduction,” Gomperts, Kramer and Tatham, Academic Press, Elsevier Science, 2002.

In tumors, the Ras-Raf-MEK-ERK pathway is thought to be the single most important pathway for the signal transduction of mitogenic signals from the plasma membrane to the nucleus. Activated Raf activates by phosphorylation the signaling kinases MEK1 and MEK2 (MEK1/2). These are dual-specificity kinases that activate the ERK family kinases, ERK1 and ERK2, by phosphorylation of both threonine and thyrosine. ERK activation results in phosphorylation and activation of ribosomal S9 kinase and transcription factors, such as c-Fos, c-Jun and c-Myc, resulting in the switching on of a number of genes involved in proliferation. A variety of growth factors, such as the erbB family, PDGF, FGF and VEGF, transmit signals through the Ras-Raf-MEK-ERK pathway. In addition, mutations in ras proto-oncogenes can result in constitutive activation of this pathway. Ras genes are mutated in approximately 30% of all human cancers, and the frequencies of ras mutations are particularly high in colon and pancreatic cancers (50% and 90%, respectively). Because of their downstream position from various mitogenic factors, MEK 1 and 2 have a central role in the transmission of proliferative signals from the plasma membrane to the nucleus. This makes these proteins an important target for cancer therapy because their inhibition would abrogate a number of different signaling pathways. Therefore, a MEK inhibitor may be active against a broad range of cancers, such as, but not limited to, breast, colon, lung, prostate, ovarian and pancreatic cancers.

2-(2-Chloro-4-iodo-phenylamino)-N-cyclopropylmethoxy-3,4-difluoro-benzamide, also known as CI-1040 is a potent and highly selective inhibitor of both MEK isoforms, MEK1 and MEK 2. Inhibition of MEK activity by CI-1040 results in a significant decrease in the levels of phosphorylated ERK1 and ERK2. This decrease produces a G1 block and impairs the growth of tumor cells, both in culture and in mice. CI-1040 has demonstrated anticancer activity against a broad spectrum of tumor types, including those of colon and pancreatic origin (Sebolt-Leopold J., et al, Blockade of the MAP kinase pathway suppresses growth of colon tumors in vivo. Nature Med. 1999; 5:810-16; and Sebolt-Leopold JS, Summary of the preclinical pharmacology of CI-1040. RR 700-00156. Jun. 27, 2000).

CI-1040 is described in PCT Publication No. WO 99/01426, which is incorporated herein by reference for its teaching of how to make CI-1040, how to formulate it into dosage forms, and how to use it for chronic oral treatment of solid tumors, such as breast, colon, prostate, skin and pancreatic cancers. CI-1040 is also described in U.S. Pat. No. 6,251,943 for use in the treatment or prevention of septic shock.

N-[(R)-2,3-Dihydroxy-propoxy]-3,4-difluoro-2-(2-fluoro-4-iodo-phenylamino)-benzamide is a potent and highly selective, inhibitor of MEK1/2, which significantly inhibits the phosphorylation of ERK1 and ERK2. N-[(R)-2,3-Dihydroxy-propoxy]-3,4-difluoro-2-(2-fluoro-4-iodo-phenylamino)-benzamide is disclosed in PCT Publication No. WO 02/06213, which is incorporated herein by reference for its teaching of how to make it, how to formulate it into dosage forms, and how to use it for chronic oral treatment of solid tumors, such as breast, colon, prostate, skin and pancreatic cancers. It is more potent and metabolically more stable than its predecessor, CI-1040.

### SUMMARY OF THE INVENTION

The present invention relates to a method for treating abnormal cell growth, preferably cancer, in a patient, preferably a human, in need of such treatment, the method comprising, administering to the patient a combination of an amount of a cell cycle inhibitor and an amount of one or more (preferably one to three, more preferably one or two, most preferably one) signal transduction inhibitors, wherein the amounts of the cell cycle inhibitor and the signal transduction inhibitor(s) when taken as a whole is therapeutically effective for treating said abnormal cell growth, preferably cancer. Cell cycle inhibitors include CDK inhibitors, Aurora Kinase inhibitors, PLK inhibitors or CHK1 inhibitors. Preferably, said cell cycle inhibitors are selective CDK inhibitors, more preferably a selective CDK-4/6 inhibitor. Most preferably, said selective CDK-4/6 inhibitor is 6-acetyl-8-cyclopentyl-5-methyl-2-(5-piperazin-1-yl-pyridin-2-ylamino)-8H-pyrido[2,3-d]pyrimidin-7-one.

Cyclin dependent kinase inhibitors as used herein refers to inhibitors that produce a direct effect on the signaling pathways that promote growth, proliferation and survival of a cell by inhibiting the activity of any of the known cyclin dependent kinases, most preferably CDK4/6. Preferred CDK4/6 inhibitors are described in International Publication WO 03/062236, published Jul. 31, 2003 and U.S. Patent Application 60/486,351 filed Jul. 11, 2003 and 60/440,805 filed Jan. 17, 2003. Examples of such inhibitors include: 8-Cyclopentyl-2-(pyridin-2-ylamino)-8H-pyrido[2,3-d]pyrimidin-7-one, 6-Bromo-8-cyclopentyl-2-(5-piperazin-1-yl-pyridin-2-ylamino)-8H-pyrido[2,3-d]pyrimidin-7-one hydrochloride, 8-Cyclopentyl-6-ethyl-2-(5-piperazin-1-yl-pyridin-2-ylamino)-8H-pyrido[2,3-d]pyrimidin-7-one hydrochloride, 8-Cyclopentyl-7-oxo-2-(5-piperazin-1-yl-pyridin-2-ylamino)-7,8-dihydro-pyrido[2,3-d]pyrimidine-6-carboxylic acid ethyl ester hydrochloride, 6-Amino-8-cyclopentyl-2-(5-piperazin-1-yl-pyridin-2-ylamino)-8H-pyrido[2,3-d]pyrimidin-7-one hydrochloride, 6-Bromo-8-cyclopentyl-2-[5-((R)-1-methy-l-pyrrolidin-2-yl)-pyridin-2-ylamino]-8H-pyrido[2,3-d]pyrimidin-7-one hydrochloride, 6-Bromo-8-cyclohexyl-2-(pyridin-2-yl-amino)-8H-pyrido[2,3-d]pyrimidin-7-one, 6-Bromo-8-cyclopentyl-2-methyl-8H-pyrido[2,3-d]pyrimidin-7-one, 6-Bromo-8-cyclopentyl-5-methyl-2-(5-piperizin-1-yl-pyridin-2-ylamino)-8H-pyrido[2,3-d]pyrimidin-7-one, 8-Cyclopentyl-6-fluoro-2-(5-piperazin-1-yl-pyridin-2-ylamino)-8H-pyrido[2,3-d]pyrimidin-7-one hydrochloride, 8-Cyclopentyl-6-methyl-2-(5-piperazin-1-yl-pyridin-2-ylamino)-8H-pyrido[2,3-d]pyrimidin-7-one hydrochloride, 8-Cyclopentyl-6-isobutoxy-2-(5-piperazin-1-yl-pyridin-2-ylamino)-8H-pyrido[2,3-d]pyrimidin-7-one hydrochloride, 6-Benzyl-8-cyclopentyl-2-(5-piperazin-1-yl-pyridin-2-ylamino)-8H-pyrido[2,3-d]pyrimidin-7-one hydrochloride, 8-Cyclopentyl-6-hydroxymethyl-2-(5-piperazin-1-yl-pyridin-2-ylamino)-8H-pyrido[2,3-d]pyrimidin-7-one hydrochloride, 2-[5-(4-tert-Butoxycarbonyl-piperazin-1-yl)-pyridin-2-ylamino]-8-cyclopentyl-5-methyl-7-oxo-7,8-dihydro-pyrido[2,3-d]pyrimidine-6-carboxylic acid ethyl ester, 6-Acetyl-8-cyclopentyl-2-(5-piperazin-1-yl-pyridin-2-ylamino)-8H-pyrido[2,3-d]pyrimidin-7-one, 6-Acetyl-8-cyclopentyl-5-methyl-2-(5-piperazin-1-yl-pyridin-2-ylamino)-8H-pyrido[2,3-d]pyrimidin-7-one, 6-Bromo-8-cyclopentyl-5-methyl-2-(pyridin-2-ylamino)-8H-pyrido[2,3-d]pyrimidin-7-one, 6-Bromo-8-cyclopentyl-2-(pyridin-2-ylamino)-8H-pyrido[2,3-d]pyrimidin-7-one, 4-Cyclopentylamino-2-(5-piperazin-1-yl-pyridin-2-ylamino)-pyrimidine-5-carbonitrile, N4-Cyclopentyl-5-nitro-N-2-(5-piperazin-1-yl-pyridin-2-yl)-pyrimidine-2,4-diamine, 4-Cyclopentylamino-2-(5-piperazin-1-yl-pyridin-2-ylamino)-pyrimidine-5-carbaldehyde, 4-Cyclopentylamino-2-(5-piperazin-1-yl-pyridin-2-ylamino)-pyrimidine-5-carboxylic acid ethyl ester, 4-Cyclopentylamino-2-(5-piperazin-1-yl-pyridin-2-ylamino)-pyrimidine-5-carboxylic acid methyl ester, [4-Cyclopentylamino-2-(5-piperazin-1-yl-pyridin-2-ylamino)-pyrimidin-5-yl]-methanol, 1-[4-Cyclopentylamino-2-(5-piperazin-1-yl-pyridin-2-ylamino)-pyrimidin-5-yl]-ethanone, 3-[4-Cyclopentylamino-2-(5-piperazin-1-yl-pyridin-2-ylamino)-pyrimidin-5-yl]-but-2-enoic acid ethyl ester, 4-Amino-2-(5-piperazin-1-yl-pyridin-2-ylamino)-pyrimidine-5-carbonitrile, 5-Nitro-N-2-(5-piperazin-1-yl-pyridin-2-yl)-pyrimidine-2,4-diamine, 4-Amino-2-(5-piperazin-1-yl-pyridin-2-ylamino)-pyrimidine-5-carbaldehyde, 4-Amino-2-(5-piperazin-1-yl-pyridin-2-ylamino)-pyrimidine-5-carboxylic acid ethyl ester, 4-Amino-2-(5-piperazin-1-yl-pyridin-2-ylamino)-pyrimidine-5-carboxylic acid methyl ester, [4-Amino-2-(5-piperazin-1-yl-pyridin-2-ylamino)-pyrimidin-5-yl]-methanol, 1-[4-Amino-2-(5-piperazin-1-yl-pyridin-2-ylamino)-pyrimidin-5-yl]-ethanone, 3-[4-Amino-2-(5-piperazin-1-yl-pyridin-2-ylamino)-pyrimidin-5-yl]-but-2-enoic acid ethyl ester, 4-Cyclopentylamino-2-(5-pyrrolidin-1-yl-pyridin-2-ylamino)-pyrimidine-5-carbonitrile, N2-[5-(3-Amino-pyrrolidin-1-yl)-pyridin-2-yl]-N-4-cyclopentyl-5-nitro-pyrimidine-2,4-diamine, 4-Cyclopentylamino-2-(5-morpholin-4-yl-pyridin-2-ylamino)-pyrimidine-5-carbaldehyde, 4-Cyclopentylamino-2-(3,4,5,6-tetrahydro-2H-[1,3′]bipyridinyl-6′-ylamino)-pyrimidine-5-carboxylic acid ethyl ester, 4-Cyclopentylamino-6-methyl-2-(5-piperazin-1-yl-pyridin-2-ylamino)-pyrimidine-5-carboxylic acid methyl ester, {2-[5-(Bis-methoxymethyl-amino)-pyridin-2-ylamino]-4-cyclopentylamino-pyrimidin-5-yl}-methanol, 1-[4-Benzylamino-2-(5-piperazin-1-yl-pyridin-2-ylamino)-pyrimidin-5-yl]-ethanone, 4-[4-Cyclopentylamino-2-(5-piperazin-1-yl-pyridin-2-ylamino)-pyrimidin-5-yl]-pent-3-en-2-one, 4-Amino-2-(pyridin-2-ylamino)-pyrimidine-5-carbonitrile, 5-Nitro-N-2-pyridin-2-yl-pyrimidine-2,4-diamine, 4-Amino-2-(pyridin-2-ylamino)-pyrimidine-5-carbaldehyde, 4-Amino-2-(pyridin-2-ylamino)-pyrimidine-5-carboxylic acid ethyl ester, 5-Bromo-N-2-(5-piperazin-1-yl-pyridin-2-yl)-pyrimidine-2,4-diamine, [4-Amino-2-(5-morpholin-4-yl-pyridin-2-ylamino)-pyrimidin-5-yl]-methanol, 1-[4-Amino-2-(5-morpholin-4-yl-pyridin-2-ylamino)-pyrimidin-5-yl]-ethanone, [6-(5-Acetyl-4-amino-pyrimidin-2-ylamino)-pyridin-3-yloxy]-acetic acid, 4-Cyclopentylamino-2-(4-hydroxymethyl-5-pyrrolidin-1-yl-pyridin-2-ylamino)-pyrimidine-5-carbonitrile, N2-[5-(3-Amino-pyrrolidin-1-yl)-6-chloro-pyridin-2-yl]-N-4-cyclopentyl-5-nitro-pyrimidine-2,4-diamine, 2-(5-Bromo-pyridin-2-ylamino)-4-cyclopentylamino-pyrimidine-5-carbaldehyde, 4-Cyclopentylamino-2-(1H-pyrrolo[3,2-b]pyridin-5-ylamino)-pyrimidine-5-carboxylic acid ethyl ester, 4-Cyclopentylamino-2-(4,6-dichloro-5-piperazin-1-yl-pyridin-2-ylamino)-6-methyl-pyrimidine-5-carboxylic acid methyl ester, 2-(2-{5-[Bis-(2-methoxy-ethyl)-amino]-pyridin-2-ylamino}-4-cyclopentylamino-pyrimidin-5-yl)-2-methyl-propan-1-ol, 1-[4-Phenylamino-2-(5-piperazin-1-yl-pyridin-2-ylamino)-pyrimidin-5-yl]-ethanone, 4-[4-(3-Hydroxy-cyclopentylamino)-2-(5-piperazin-1-yl-pyridin-2-ylamino)-pyrimidin-5-yl]-pent-3-en-2-one, 4-[5-Cyano-2-(pyridin-2-ylamino)-pyrimidin-4-ylamino]-cyclohexanecarboxylic acid, 2-(4-Amino-5-nitro-pyrimidin-2-ylamino)-isonicotinic acid, 4-Amino-6-methyl-2-(pyridin-2-ylamino)-pyrimidine-5-carbaldehyde, 5-Iodo-N-2-pyridin-2-yl-pyrimidine-2,4-diamine, N-[5-Bromo-2-(5-piperazin-1-yl-pyridin-2-ylamino)-pyrimidin-4-yl]-acrylamide, N2-(5-piperazin-1-yl-pyridin-2-yl)-5-prop-1-ynyl-pyrimidine-2,4-diamine, 5-[2-(4-Fluoro-phenyl)-ethyl]-N-2-(5-piperazin-1-yl-pyridin-2-yl)-pyrimidine-2,4-diamine, [6-(4-Amino-5-propenyl-pyrimidin-2-ylamino)-pyridin-3-yloxy]-acetic acid, 5-Bromo-N-4-cyclopentyl-N-2-(5-pyrrolidin-1-yl-pyridin-2-yl)-pyrimidine-2,4-diamine, N2-[5-(3-Amino-pyrrolidin-1-yl)-6-chloro-pyridin-2-yl]-5-bromo-N-4-cyclopentyl-pyrimidine-2,4-diamine, 5-Bromo-N-4-cyclopentyl-N-2-(5-piperazin-1-yl-pyridin-2-yl)-pyrimidine-2,4-diamine, 5-Bromo-N-4-cyclopentyl-N-2-(1H-pyrrolo[3,2-b]pyridin-5-yl)-pyrimidine-2,4-diamine, 5-Bromo-N4-cyclopentyl-N-2-(4,6-dichloro-5-piperazin-1-yl-pyridin-2-yl)-6-methyl-pyrimidine-2,4-diamine, N2-{5-[Bis-(2-methoxy-ethyl)-amino]-pyridin-2-yl}-5-bromo-N-4-cyclopentyl-pyrimidine-2,4-diamine, 5-Bromo-N-4-phenyl-N-2-(5-piperazin-1-yl-pyridin-2-yl)-pyrimidine-2,4-diamine, 3-[5-Bromo-2-(5-piperazin-1-yl-pyridin-2-ylamino)-pyrimidin-4-ylamino]-cyclopentanol, N4-Cyclopentyl-5-iodo-N-2-(5-pyrrolidin-1-yl-pyridin-2-yl)-pyrimidine-2,4-diamine, N2-[5-(3-Amino-pyrrolidin-1-yl)-6-chloro-pyridin-2-yl]-N-4-cyclopentyl-5-iodo-pyrimidine-2,4-diamine, N4-Cyclopentyl-5-iodo-N-2-(5-piperazin-1-yl-pyridin-2-yl)-pyrimidine-2,4-diamine, N4-Cyclopentyl-5-iodo-N-2-(1H-pyrrolo[3,2-b]pyridin-5-yl)-pyrimidine-2,4-diamine, 4-[6-(5-Bromo-4-cyclopentylamino-pyrimidin-2-ylamino)-pyridin-3-yl]-piperazine-1-carboxylic acid tert-butyl ester, 4-[6-(4-Cyclopentylamino-5-formyl-pyrimidin-2-ylamino)-pyridin-3-yl]-piperazine-1-carboxylic acid tert-butyl ester, 4-[6-(5-Acetyl-4-cyclopentylamino-pyrimidin-2-ylamino)-pyridin-3-yl]-piperazine-1-carboxylic acid tert-butyl ester, 2-[5-(4-tert-Butoxycarbonyl-piperazin-1-yl)-pyridin-2-ylamino]-4-cyclopentylamino-pyrimidine-5-carboxylic acid ethyl ester, N-Cyclopentyl-N′-(5-piperazin-1-yl-pyridin-2-yl)-pyrimidine-4,6-diamine, N-Isopropyl-N′-(5-piperazin-1-yl-pyridin-2-yl)-pyrimidine-4,6-diamine, 4-[6-(6-Cyclopentylamino-pyrimidin-4-ylamino)-pyridin-3-yl]-piperazine-1-carboxylic acid tert-butyl ester, N-[5-(3-Amino-pyrrolidin-1-yl)-pyridin-2-yl]-N′-cyclopentyl-pyrimidine-4,6-diamine, 4-{6-[4-Cyclopentylamino-5-(1-methyl-3-oxo-but-1-enyl)-pyrimidin-2-ylamino]-pyridin-3-yl}-piperazine-1-carboxylic acid tert-butyl ester, N-Cyclopentyl-N′-(5-piperazin-1-yl-pyridin-2-yl)-[1,3,5]triazine-2,4-diamine, 1-[4-Cyclopentylamino-2-(5-piperazin-1-yl-pyridin-2-ylamino)-pyrimidin-5-yl]-ethanone, 5-Bromo-N4-cyclopentyl-N-2-(5-piperazin-1-yl-pyridin-2-yl)-pyridine-2,4-diamine, 4-Cyclopentylamino-6-(5-piperazin-1-yl-pyridin-2-ylamino)-nicotinonitrile, N4-Cyclopentyl-5-nitro-N-2-(5-piperazin-1-yl-pyridin-2-yl)-pyridine-2,4-diamine, 4-Cyclopentylamino-6-(5-piperazin-1-yl-pyridin-2-ylamino)-pyridine-3-carbaldehyde, 4-Cyclopentylamino-6-(5-piperazin-1-yl-pyridin-2-ylamino)-nicotinic acid ethyl ester, 4-Cyclopentylamino-6-(5-piperazin-1-yl-pyridin-2-ylamino)-nicotinic acid methyl ester, [4-Cyclopentylamino-6-(5-piperazin-1-yl-pyridin-2-ylamino)-pyridin-3-yl]-methanol, 1-[4-Cyclopentylamino-6-(5-piperazin-1-yl-pyridin-2-ylamino)-pyridin-3-yl]-ethanone, 3-[4-Cyclopentylamino-6-(5-piperazin-1-yl-pyridin-2-ylamino)-pyridin-3-yl]-but-2-enoic acid ethyl ester, (5-Cyclopentyl-5,6-dihydro-pyrido[2,3-e][1,2,4]triazin-3-yl)-(5-piperazin-1-yl-pyridin-2-yl)-amine, (8-Cyclopentyl-7-methoxy-quinazolin-2-yl)-(5-piperazin-1-yl-pyridin-2-yl)-amine, (8-Cyclopentyl-7-methoxy-pyrido[3,2-d]pyrimidin-2-yl)-(5-piperazin-1-yl-pyridin-2-yl)-amine, 6-Acetyl-8-cyclopentyl-2-(5-piperazin-1-yl-pyridin-2-ylamino)-8H-pyridin-7-one, 3-Acetyl-1-cyclopentyl-7-(5-piperazin-1-yl-pyridin-2-ylamino)-1H-pyrido[3,4-b]pyrazin-2-one, 1-Cyclopentyl-3-ethyl-4-methyl-7-(5-piperazin-1-yl-pyridin-2-ylamino)-3,4-dihydro-1H-pyrimido[4,5-d]pyrimidin-2-one, 1-Cyclopentyl-3-ethyl-4-methyl-7-(5-piperazin-1-yl-pyridin-2-ylamino)-3,4-dihydro-1H-pyrido[4,3-d]pyrimidin-2-one, 3-Acetyl-1-cyclopentyl-4-methyl-7-(5-piperazin-1-yl-pyridin-2-ylamino)-1H-[1,6]naphthyridin-2-one, (9-Isopropyl-6-methyl-9H-purin-2-yl)-(5-piperazin-1-yl-pyridin-2-yl)-amine, 2-[9-Isopropyl-6-(5-piperazin-1-yl-pyridin-2-ylamino)-9H-purin-2-ylamino]-ethanol, N2-(4-Amino-cyclohexyl)-9-cyclopentyl-N-6-(5-piperazin-1-yl-pyridin-2-yl)-9H-purine-2,6-diamine, 2-[9-Isopropyl-6-(5-piperazin-1-yl-pyridin-2-ylamino)-9H-purin-2-ylamino]-3-methyl-butan-1-ol, (1-Isopropyl-4-methyl-1H-pyrazolo[3,4-d]pyrimidin-6-yl)-(5-piperazin-1-yl-pyridin-2-yl)-amine, 2-[1-Isopropyl-4-(5-piperazin-1-yl-pyridin-2-ylamino)-1H-pyrazolo[3,4-d]pyrimidin-6-ylamino]-ethanol, N6-(4-Amino-cyclohexyl)-1-cyclopentyl-N-4-(5-piperazin-1-yl-pyridin-2-yl)-1H-pyrazolo[3,4-d]pyrimidine-4,6-diamine, 2-[1-Isopropyl-4-(5-piperazin-1-yl-pyridin-2-ylamino)-1H-pyrazolo[3,4-d]pyrimidin-6-ylamino]-3-methyl-butan-1-ol, 5-Cyclopentyl-7-(1-hydroxy-ethyl)-8-methyl-3-(5-piperazin-1-yl-pyridin-2-ylamino)-5H-pyrido[3,2-c]pyridazin-6-one, 5-Cyclopentyl-8-methyl-3-(5-piperazin-1-yl-pyridin-2-ylamino)-5H-pyrido[3,2-c]pyridazin-6-one, 7-Benzyl-5-cyclopentyl-3-(5-piperazin-1-yl-pyridin-2-ylamino)-5H-pyrido[3,2-c]pyridazin-6-one, [5-(1,1-Dioxo-1l6-thiomorpholin-4-yl)-pyridin-2-yl]-(4-isopropyl-3-methoxy-2-methyl-[1,7]naphthyridin-6-yl)-amine, (2-Ethyl-4-isopropyl-3-methoxy-[1,7]naphthyridin-6-yl)-pyridin-2-yl-amine, (2,4-Diisopropyl-3-methoxy-[1,7]naphthyridin-6-yl)-(5-isopropenyl-pyridin-2-yl)-amine, [4-(2-Ethylamino-pyridin-4-yl)-pyrimidin-2-yl]-(5-piperazin-1-yl-pyridin-2-yl)-amine, [4-(5-Ethyl-2-methylamino-pyridin-4-yl)-pyrimidin-2-yl]-(5-morpholin-4-yl-pyridin-2-yl)-amine, [5-Methoxy-4-(2-methylamino-pyridin-4-yl)-pyrimidin-2-yl]-(5-morpholin-4-yl-pyridin-2-yl)-amine, and 5-Fluoro-N-4-isopropyl-N-2-(5-piperazin-1-yl-pyridin-2-yl)-pyrimidine-2,4-diamine. Other CDK inhibitors of interest include AG-24322, R-roscovitine, CYC202 (a CDK2 selective inhibitor), flavopiridol (NSC 649890, HMR 1275) (a non-selective CDK inhibitor), NU6102 (a CDK1/2 selective inhibitor), alsterpaullone (a CDK1/B inhibitor), indirubin-3′-monoxime (a CDK1/B/5 inhibitor) BMS 387032 (a CDK(1/B)(2/E)(4/D) inhibitor) and 7-hydroxystaurosporine (UCN-01, NSC 638850). Other specific CDK inhibitors are described in EP1250353, WO 02/96888, WO 03/076437, WO 03/76436, WO 03/76434, WO 01/64368; U.S. Provisional Application No. 60/491,474 and U.S. Provisional Application No. 60/491,474.

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates a CDK protein. Upregulation includes: over expressing CDK4/6 or cyclin D, under expressing p16 or mutation of CDK4/6. See D. Fry, Curr. Opin. Oncologic, Endocrine & Metabolic Invest. Drugs. 2000, 2, 40-59.

Signal transduction inhibitors (STI) as used herein refers to inhibitors that produce a direct effect on the signaling pathways that promote growth, proliferation and survival of a cell. One skilled in the art will appreciate that STI's so defined includes Growth Factors and mitogen stimulated kinase signal transduction pathway inhibitors. Such inhibitors also include small molecules, antibodies, and antisense molecules. Signal transduction inhibitors as described herein comprise tyrosine kinase inhibitors, serine/threonine kinase inhibitors, dual specificity kinase inhibitors, lipid kinase inhibitors, histone deacetylase inhibitors, Bc12 inhibitors, p53 inhibitors, MDMZ inhibitors, Ras inhibitors and Hsp 90 inhibitors.

One embodiment of the invention is directed to those combinations of an amount of a cell cycle inhibitor with an amount of a dual specificity kinase inhibitor such as a MEK inhibitor, wherein the amounts of the cell cycle inhibitor and the dual specificity kinase inhibitor when taken as a whole is therapeutically effective for treating said abnormal cell growth, preferably cancer.

Dual specificity kinase inhibitors refers to inhibitors that produce a direct effect on the signaling pathways that promote growth, proliferation and survival of a cell by inhibiting the activity of multiple tyrosine kinases and serine/threonine kinases, such as MEK1 or MEK2. Examples of such most preferred MEK inhibitors include 2-(2-chloro-4-iodo-phenylamino)-N-cyclopropylmethoxy-3,4-difluoro-benzamide and N-[(R)-2,3-Dihydroxy-propoxy]-3,4-difluoro-2-(2-fluoro-4-iodo-phenylamino)-benzamide. 2-(2-chloro-4-iodo-phenylamino)-N-cyclopropylmethoxy-3,4-difluoro-benzamide and N-[(R)-2,3-Dihydroxy-propoxy]-3,4-difluoro-2-(2-fluoro-4-iodo-phenylamino)-benzamide are selective MEK 1 and MEK 2 inhibitors. Selective MEK 1 or MEK 2 inhibitors are those compounds which inhibit the MEK 1 or MEK 2 enzymes without substantially inhibiting other enzymes such as MKK3, ERK, PKC, Cdk2A, phosphorylase kinase, EGF and PDGF receptor kinases, and C-src.

A most preferred embodiment of the invention is directed to a combination of the CDK inhibitor 6-acetyl-8-cyclopentyl-5-methyl-2-(5-piperazin-1-yl-pyridin-2-ylamino)-8H-pyrido[2,3-d]pyrimidin-7-one and a MEK inhibitor, wherein the amounts of the cell cycle inhibitor and the MEK inhibitor when taken as a whole is therapeutically effective for treating said abnormal cell growth, preferably cancer.

Examples of MEK inhibitors according to the present invention include, but are not limited to the MEK inhibitors disclosed in the following PCT Publications: WO 99/01426, WO 99/01421, WO 00/42002, WO 00/42022, WO 00/41994, WO 00/42029, WO 00/41505, WO 00/42003, WO 01/68619, and WO 02/06213.

Another MEK inhibitor embodiment of the invention includes the compound 1,4-diamino-2,3-dicyano-1,4-bis[2-aminophenylthio]butadiene (U-0126).

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates a MEK protein.

In another preferred embodiment of the present invention, certain small molecule combinations of CDK inhibitors and MEK inhibitors can additionally be combined with antibody STI's such as Herceptin (trastuzumab), Erbitux (C225), and small molecule STI's such as Iressa (gefitinib) and Tarceva (erlotinib).

Another embodiment of the invention is directed to those combinations of an amount of a cell cycle inhibitor, (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one), with an amount of serine/threonine kinase inhibitor(s) (preferably one inhibitor), wherein the amounts of the cell cycle inhibitor and the serine/threonine kinase inhibitor(s) when taken as a whole is therapeutically effective for treating said abnormal cell growth, preferably cancer. More preferably such serine/threonine kinase inhibitors include Raf kinase inhibitors, Akt inhibitors and mTOR inhibitors.

Another embodiment of the invention is directed to a combination of an amount of an amount of a cell cycle inhibitor (preferably a selective CDK inhibitor, more preferably a selective CDK4/6 inhibitor, (most preferably 6-acetyl-8-cyclopentyl-5-methyl-2-(5-piperazin-1-yl-pyridin-2-ylamino)-8H-pyrido[2,3-d]pyrimidin-7-one) and an amount of a Raf Kinase inhibitor, wherein the amounts of the cell cycle inhibitor and the Raf Kinase inhibitor when taken as a whole is therapeutically effective for treating said abnormal cell growth, preferably cancer. Raf kinase inhibitors as used herein refers to inhibitors that produce a direct effect on the signaling pathways that promote growth, proliferation and survival of a cell by inhibiting the function of the Raf protein. A preferred example of such an inhibitors is BAY 43-9006. Other Raf kinase inhibitors include those described in WO 03/68223, published Aug. 21, 2003, WO 03/82272, published Oct. 9, 2003, WO 03/22840, published Mar. 20, 2003, WO 03/22838, published Mar. 20, 2003, WO 03/22837, published Mar. 20, 2003, WO 03/22836, published Mar. 20, 2003, and WO 03/22833, published Mar. 20, 2003.

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates a Raf protein. In a particular embodiment, the level of expression of Raf is +2 or +3 on a four-value scale that ranges from 0 (normal) to +1 to +2 to +3. A value of +3 is associated with highly aggressive tumors.

Another embodiment of the invention is directed to a combination of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) and an Akt inhibitor wherein the amounts of the cell cycle inhibitor and the Akt inhibitor(s) when taken as a whole is therapeutically effective for treating said abnormal cell growth, preferably cancer. Akt inhibitors as used herein refers to inhibitors that produce a direct effect on the signaling pathways that promote growth, proliferation and survival of a cell by inhibiting the function of the Akt protein. Examples of such Akt inhibitors include those compounds described in European Patent Publication EP 1379251, and International Publications WO 03/86403, WO 03/86394 and WO03/86279, all published Oct. 23, 2003.

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates an Akt protein. In a particular embodiment, the level of expression of Akt is +2 or +3 on a four-value scale that ranges from 0 (normal) to +1 to +2 to +3. A value of +3 is associated with highly aggressive tumors.

Another embodiment of the invention is directed to a combination of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) and an mTOR inhibitor, wherein the amounts of the cell cycle inhibitor and the mTOR inhibitor(s) when taken as a whole is therapeutically effective for treating said abnormal cell growth, preferably cancer. mTOR inhibitors as used herein refers to inhibitors that produce a direct effect on the signaling pathways that promote growth, proliferation and survival of a cell by inhibiting the function of the mTOR protein. Examples of such inhibitors include rapamycins, preferably rapamycin, CCI 779, Rad001 and Arry 142886.

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates an m-TOR protein. In a particular embodiment, the level of expression of m-TOR is +2 or +3 on a four-value scale that ranges from 0 (normal) to +1 to +2 to +3. A value of +3 is associated with highly aggressive tumors.

Another embodiment of the invention is directed to those combinations of an amount of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) with an amount of tyrosine kinase inhibitor(s) (preferably one inhibitor), wherein the amounts of the cell cycle inhibitor and the tyrosine kinase inhibitor(s) when taken as a whole is therapeutically effective for treating said abnormal cell growth, preferably cancer. More preferably such tyrosine kinase inhibitors include bcr-abl tyrosine kinase inhibitors, PDGFR inhibitors, c-Kit inhibitors, erbB inhibitors, VEGF-R inhibitors, FGFR inhibitors, TGFβR, Src, and IGF1-R inhibitors.

An embodiment of the invention is directed to a combination of an amount of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) and an amount of a bcr-abl tyrosine kinase inhibitor, wherein the amounts of the cell cycle inhibitor and the bcr-abl tyrosine kinase inhibitor when taken as a whole is therapeutically effective for treating said abnormal cell growth, preferably cancer. Bcr-abl tyrosine kinase inhibitors as used herein refers to inhibitors that produce a direct effect on the signaling pathways that promote growth, proliferation and survival of a cell by inhibiting the function of the bcr-abl protein. Examples of such inhibitors include Gleevec.

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates a bcr-abl protein. In a particular embodiment, the level of expression of bcr-abl is +2 or +3 on a four-value scale that ranges from 0 (normal) to +1 to +2 to +3. A value of +3 is associated with highly aggressive tumors.

Another embodiment of the invention is directed to a combination of an amount of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) and an amount of a PDGFR inhibitor, wherein the amounts of the cell cycle inhibitor and the PDGFR inhibitor when taken as a whole is therapeutically effective for treating said abnormal cell growth, preferably cancer. PDGFR inhibitors as used herein refers to inhibitors that produce a direct effect on the signaling pathways that promote growth, proliferation and survival of a cell by inhibiting the function of the PDGFR protein. Examples of such inhibitors include CP-868, 596, ST-1571, PTK-787 and PKC-412. PDGFR inhibitors include the compounds disclosed and claimed in U.S. patent application Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following U.S. Provisional Patent Applications: 60/168,207 (filed Nov. 30, 1999); 60/170,119 (filed Dec. 10, 1999); 60/177,718 (filed Jan. 21, 2000); 60/168,217 (filed Nov. 30, 1999), 60/200,834 (filed May 1, 2000), 60/406,524 (filed Aug. 28, 2002) and 60/417,074 (filed Oct. 8, 2002). PDGFR inhibitors are also disclosed and claimed in International Patent Publication WO2001/40217, published Jun. 7, 2001

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates a PDGFR receptor. In a particular embodiment, the level of expression of PDGFR is +2 or +3 on a four-value scale that ranges from 0 (normal) to +1 to +2 to +3. A value of +3 is associated with highly aggressive tumors.

Another embodiment of the invention is directed to a combination of an amount of a CDK inhibitor (preferably 6-acetyl-8-cyclopentyl-5-methyl-2-(5-piperazin-1-yl-pyridin-2-ylamino)-8H-pyrido[2,3-d]pyrimidin-7-one) and an amount of a c-Kit inhibitor, wherein the amounts of the CDK inhibitor and the c-Kit inhibitor when taken as a whole is therapeutically effective for treating said abnormal cell growth, preferably cancer. c-Kit inhibitors as used herein refers to inhibitors that produce a direct effect on the signaling pathways that promote growth, proliferation and survival of a cell by inhibiting the c-Kit protein. Examples of such inhibitors include those compounds described in International Patent Publications WO 03/028711, published Apr. 10, 2003 and WO 03/002114, published Jan. 9, 2003.

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates a c-Kit protein. In a particular embodiment, the level of expression of c-Kit is +2 or +3 on a four-value scale that ranges from 0 (normal) to +1 to +2 to +3. A value of +3 is associated with highly aggressive tumors.

Another embodiment of the invention is directed to a combination of an amount of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) and an amount of an erbB inhibitor, including an erbB-1 inhibitor, erbB-2 inhibitor or an erbB1/erbB2 inhibitor, wherein the amounts of the cell cycle inhibitor and the erbB inhibitor when taken as a whole is therapeutically effective for treating said abnormal cell growth, preferably cancer. ErbB inhibitors as used herein refers to inhibitors that produce a direct effect on the signaling pathways that promote growth, proliferation and survival of a cell by inhibiting the function of erbB proteins. Examples of such erbB, erbB2 or erbB1/erbB2 inhibitors include Herceptin (trastuzumab), Erbitux, Iressa (gefitinib), Tarceva (erlotinib), EKB-569, PKI-166, GW-572016, E-2-methoxy-N-(3-{4-[3-methyl-4-(6-methyl-pyridin-3-yloxy)-phenylamino]-quinazolin-6-yl}-allyl)-acetamide and CI-1033, preferably E-2-methoxy-N-(3-{4-[3-methyl-4-(6-methyl-pyridin-3-yloxy)-phenylamino]-quinazolin-6-yl}-allyl)-acetamide and CI-1033, more preferably E-2-methoxy-N-(3-{4-[3-methyl-4-(6-methyl-pyridin-3-yloxy)-phenylamino]-quinazolin-6-yl}-allyl)-acetamide. ErbB2 receptor inhibitors include such compounds as E-2-methoxy-N-(3-{4-[3-methyl-4-(6-methyl-pyridin-3-yloxy)-phenylamino]-quinazolin-6-yl}-allyl)-acetamide, GW-282974 (Glaxo Wellcome plc), and the monoclonal antibodies AR-209 (Aronex Pharmaceuticals Inc. of The Woodlands, Tex., USA) and 2B-1 (Chiron). Such erbB2 inhibitors include those described in WO 03/50108 (published Jun. 19, 2003), WO 01/98277 (published Dec. 27, 2001), WO 00/44728 (published Aug. 3, 2000), WO 98/02434 (published Jan. 22, 1998), WO 99/35146 (published Jul. 15, 1999), WO 99/35132 (published Jul. 15, 1999), WO 98/02437 -(published Jan. 22, 1998), WO 97/13760 (published Apr. 17, 1997), WO 95/19970 (published Jul. 27, 1995), U.S. Pat. No. 5,587,458 (issued Dec. 24, 1996), U.S. Pat. No. 5,877,305 (issued Mar. 2, 1999) and U.S. Pat. No. 6,284,764 (issued Sep. 4, 2001), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety. Other erbb2 receptor inhibitors include TAK-165 (Takeda) and GW-572016 (Glaxo-Wellcome). Pan-erBB inhibitors (active against erbB1 and erB2) are described in U.S. Pat. No. 5,464,861 issued Nov. 17, 1995, U.S. Pat. No. 5,654,307 issued Aug. 5, 1997, U.S. Pat. No. 6,344,459 issued Feb. 5, 2002 U.S. Pat. No. 6,127,374 issued Oct. 3, 2000, U.S. Pat. No. 6,153,617 issued Nov. 28, 2000, U.S. Pat. No. 6,344,455 issued Feb. 5, 2002, U.S. Pat. No. 6,664,390 issued Dec. 16, 2003 and International Publication WO 02/00630 published Jan. 3, 2002.

One skilled in the art will appreciate that historically anti-cancer agents have been used for specific tumors, e.g. brain, breast, lung, such as non-small cell lung, ovarian, pancreatic, prostate, renal, colorectal, cervical, acute leukemia, and gastric cancer. The combinations of the present invention are directed to the new understanding of the molecular basis for cancer including in some cases the over expression of certain kinases. Although it is possible that a sole kinase is upregulated or overexpressed in a cancerous state, the present inventors have discovered that it is surprisingly efficacious to complement the suppression of one pathway by suppression of other kinase pathways. Thus, in yet another aspect of the present invention, the method of the invention comprises treatment of a cancer that upregulates an erbB2 protein. In a particular embodiment, the level of expression of erbB2 is +2 or +3 on a four-value scale that ranges from 0 (normal) to +1 to +2 to +3. A value of +3 is associated with highly aggressive tumors.

Specific preferred erbB2 compounds of the combinations of the present invention include those including one or more of the following compounds:
(±)-[3-Methyl-4-(pyridin-3-yloxy)-phenyl]-(6-piperidin-3-ylethynyl-quinazolin-4-yl)-amine;
2-Methoxy-N-(3-{4-[3-methyl-4-(pyridin-3-yloxy)-phenylamino]-quinazolin-6-yl}-prop-2-ynyl)-acetamide;
(±)-[3-Methyl-4-(6-methyl-pyridin-3-yloxy)-phenyl]-(6-piperidin-3-ylethynyl-quinazolin-4-yl)-amine;
[3-Methyl-4-(6-methyl-pyridin-3-yloxy)-phenyl]-(6-piperidin-4-ylethynyl-quinazolin-4-yl)-amine;
2-Methoxy-N-(3-{4-[3-methyl-4-(6-methyl-pyridin-3-yloxy)-phenylamino]-quinazolin-6-yl}-prop-2-ynyl)-acetamide;
2-Fluoro-N-(3-{4-[3-methyl-4-(6-methyl-pyridin-3-yloxy)-phenylamino]-quinazolin-6-yl}-prop-2-ynyl)-acetamide;
E-2-Methoxy-N-(3-{4-[3-methyl-4-(6-methyl-pyridin-3-yloxy)-phenylamino]-quinazolin-6-yl}-allyl)-acetamide;
[3-Methyl-4-(pyridin-3-yloxy)-phenyl]-(6-piperidin-4-ylethynyl-quinazolin-4-yl)-amine;
2-Methoxy-N-(1-{4-[3-methyl-4-(6-methyl-pyridin-3-yloxy)-phenylamino]-quinazolin-6-ylethynyl}-cyclopropyl)-acetamide;
E-N-(3-{4-[3-Chloro-4-(6-methyl-pyridin-3-yloxy)-phenylamino]-quinazolin-6-yl}-allyl)-2-methoxy-acetamide;
N-(3-{4-[3-Chloro-4-(6-methyl-pyridin-3-yloxy)-phenylamino]-quinazolin-6-yl}-prop-2-ynyl)-acetamide;
N-(3-{4-[3-Methyl-4-(6-methyl-pyridin-3-yloxy)-phenylamino]-quinazolin-6-yl}-prop-2-ynyl)-acetamide;
E-N-(3-{4-[3-Chloro-4-(6-methyl-pyridin-3-yloxy)-phenylamino]-quinazolin-6-yl}-allyl)-acetamide;
E-2-Ethoxy-N-(3-{4-[3-methyl-4-(6-methyl-pyridin-3-yloxy)-phenylamino]-quinazolin-6-yl}-allyl)-acetamide;
1-Ethyl-3-(3-{4-[3-methyl-4-(6-methyl-pyridin-3-yloxy)-phenylamino]-quinazolin-6-yl}-prop-2-ynyl)-urea;
piperazine-1-carboxylic acid (3-{4-[3-methyl-4-(6-methyl-pyridin-3-yloxy)-phenylamino]-quinazolin-6-yl}-prop-2-ynyl)-amide;
(±)-2-Hydroxymethyl-pyrrolidine-1-carboxylic acid (3-{4-[3-methyl-4-(6-methyl-pyridin-3-yloxy)-phenylamino]-quinazolin-6-yl}-prop-2-ynyl)-amide;
2-Dimethylamino-N-(3-{4-[3-methyl-4-(pyridin-3-yloxy)-phenylamino]-quinazolin-6-yl}-prop-2-ynyl)-acetamide;
E-N-(3-{4-[3-Methyl-4-(6-methyl-pyridin-3-yloxy)-phenylamino]-quinazolin-6-yl}-allyl)-methanesulfonamide;
Isoxazole-5-carboxylic acid (3-{4-[3-methyl-4-(6-methyl-pyridin-3-yloxy)-phenylamino]-quinazolin-6-yl}-prop-2-ynyl)-amide;
1-(1,1-Dimethyl-3-{4-[3-methyl-4-(6-methyl-pyridin-3-yloxy)-phenylamino]-quinazolin-6-yl}-prop-2-ynyl)-3-ethyl-urea;
   and the pharmaceutically acceptable salts, prodrugs and solvates of the foregoing compounds.

In another preferred embodiment of the present invention, certain small molecule combinations of CDK inhibitors and erbB inhibitors can additionally be combined with antibody STI's such as Herceptin (trastuzumab), Erbitux, and small molecule STIs such as Iressa (gefitinib) and Tarceva (erlotinib).

Another embodiment of the invention is directed to a combination of an amount of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) and an amount of a VEGF-R inhibitor, wherein the amounts of the cell cycle inhibitor and the VEGF-R inhibitor when taken as a whole is therapeutically effective for treating said cancer. VEGF-R inhibitors as used herein refers to inhibitors that produce a direct effect on the signaling pathways that promote growth, proliferation and survival of a cell by inhibiting the function of the VEGFR protein. Examples of such inhibitors include CP-547,632 (3-(4-Bromo-2,6-difluoro-benzyloxy)-5-[3-(4-pyrrolidin-1-yl-butyl)-ureido]-isothiazole-4-carboxylic acid amide hydrochloride), PTK 787, ZD 6474, AG-13736, AG-28262 and PKC 412. Preferred VEGF inhibitors include, for example, SU-5416 and SU-6668 (formerly Sugen Inc. of South San Francisco, Calif., USA, now Pfizer Inc.) and CP-547,632. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. WO 01/02369 (published Jan. 11, 2001); U.S. Provisional Application No. 60/491,771 (filed Jul. 31, 2003); U.S. Provisional Application No. 60/460,695 (filed Apr. 3, 2003); and WO 03/106462A1 (published Dec. 24, 2003). Other examples of VEGF inhibitors are disclosed in International Patent Publications WO 99/62890 published Dec. 9, 1999, WO 01/95353 published Dec. 13, 2001 and WO 02/44158 published Jun. 6, 2002. Other examples of some specific VEGF inhibitors are IM862 (Cytran Inc. of Kirkland, Wash., USA); Avastin, an anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

Another embodiment of the invention is directed to a combination of an amount of a cell cycle inhibitor (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) with an amount of an inhibitor of Growth Factor Signaling, wherein the amounts of the cell cycle inhibitor and the inhibitor of Growth Factor when taken as a whole is therapeutically effective for treating said abnormal cell growth, preferably cancer. Examples of inhibitors of Growth Factor Signaling include small molecules and monoclonal antibodies. Such antibodies may bind to either the receptor or to the Growth Factor. Avastin is an example of a monoclonal antibody that binds to the Growth Factor and prevents its binding to the receptor.

Another embodiment of the invention is directed to those combinations of cell cycle inhibitors (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-8H-pyrido[2,3-d]pyrimidin-7-one) with an inhibitor selected from the group consisting of lipid kinase inhibitors, histone deacetylase inhibitors Bc12 inhibitors, p53 inhibitors, MDMZ inhibitors, Ras inhibitors and Hsp 90 inhibitors, wherein the amounts of the cell cycle inhibitor and an inhibitor of lipid kinase inhibitors, histone deacetylase inhibitors Bc12 inhibitors, p53 inhibitors, MDMZ inhibitors, Ras inhibitors or Hsp 90 inhibitors when taken as a whole is therapeutically effective for treating said abnormal cell growth, preferably cancer. More preferably such other kinase inhibitors include Hsp 90 inhibitors, K-Ras inhibitors, PI3 kinase inhibitors, HDAC inhibitors, Bc12 inhibitors, TGFbeta-R inhibitors, Chk1 inhibitors, Wee1 inhibitors, PLK inhibitors, Src inhibitors, PDK inhibitors, PKC inhibitors and p70S6K inhibitors.

Another embodiment of the invention is directed to a combination of an amount of a CDK inhibitor, (preferably 6-acetyl-8-cyclopentyl-5-methyl-2-(5-piperazin-1-yl-pyridin-2-ylamino)-8H-pyrido[2,3-d]pyrimidin-7-one) and an amount of a Hsp 90 inhibitor, wherein the amounts of the CDK inhibitor and the Hsp 90 inhibitor when taken as a whole is therapeutically effective for treating said abnormal cell growth, preferably cancer. Hsp 90 inhibitors as used herein refers to inhibitors that produce a direct effect on the signaling pathways that promote growth, proliferation and survival of a cell by inhibiting the function of the Hsp 90 protein. Examples of such inhibitors include those compounds described in International Publications WO 03/089006; WO 03/041643; and WO 02/036171.

Another embodiment of the invention is directed to a combination of an amount of a CDK inhibitor, (preferably 6-acetyl-8-cyclopentyl-5-methyl-2-(5-piperazin-1-yl-pyridin-2-ylamino)-8H-pyrido[2,3-d]pyrimidin-7-one) and an amount of a K-Ras inhibitor, wherein the amounts of the CDK inhibitor and the K-Ras inhibitor when taken as a whole is therapeutically effective for treating said abnormal cell growth, preferably cancer. K-Ras inhibitors as used herein refers to inhibitors that produce a direct effect on the signaling pathways that promote growth, proliferation and survival of a cell by inhibiting the function of the K-Ras protein. Examples of such inhibitors include those described in U.S. Pat. No. 6,436,700; and U.S. Patent Publication 20030153521.

Another embodiment of the invention is directed to a combination of an amount of a CDK inhibitor, (preferably 6-acetyl-8-cyclopentyl-5-methyl-2-(5-piperazin-1-yl-pyridin-2-ylamino)-81+pyrido[2,3-d]pyrimidin-7-one) and an amount of a PI3 kinase inhibitor, wherein the amounts of the cell cycle inhibitor and the PI3 kinase inhibitor when taken as a whole is therapeutically effective for treating said abnormal cell growth, preferably cancer. PI3 kinase inhibitors as used herein refers to inhibitors that produce a direct effect on the signaling pathways that promote growth, proliferation and survival of a cell by inhibiting the function of the PI3 protein. Examples of such inhibitors include WO 01/81346; WO 01/53266; and WO 01/83456. U.S. patent application Ser. No. 10/730,680 (filed Dec. 8, 2003); U.S. patent application Ser. No. 10/743,852 (filed Dec. 22, 2003); U.S. Provisional Patent Application No. 60/475,970 (filed Jun. 5, 2003); U.S. Provisional Patent Application No. 60/475,992 (filed Jun. 5, 2003); U.S. Provisional Patent Application No. 60/476,073 (filed Jun. 5, 2003); U.S. Provisional Patent Application No. 60/476,251 (filed Jun. 5, 2003); U.S. Provisional Patent Application No. 60/475,971 (filed Jun. 5, 2003); and U.S. Provisional Patent Application No. 60/476,057 (filed Jun. 5, 2003).

Another embodiment of the invention is directed to those combinations of an amount of cell cycle inhibitors (preferably a selective CDK inhibitor, more preferably a selective CDK4/6 inhibitor, most preferably 6-acetyl-8-cyclopentyl-5-methyl-2-(5-piperazin-1-yl-pyridin-2-ylamino)-81+pyrido[2,3-d]pyrimidin-7-one) with an amount of a multi-targeted kinase inhibitor, wherein the amounts of the cell cycle inhibitor and the multi-targeted kinase inhibitor when taken as a whole is therapeutically effective for treating said abnormal cell growth, preferably cancer. More preferably such multi-targeted kinase inhibitors include inhibitors with multiple activities against any of the aforementioned kinase pathways. One embodiment of such multi-targeted kinase inhibitors are those agents with activity against PDGFR, VEGFR and FGFR (such as SU11248). Another embodiment of such multi-targeted kinase inhibitors are those agents with activity against PDGFR, c-Kit and brc-abl (such as Gleevec).

Another embodiment of the invention is directed to a combination of an amount of a CDK inhibitor, (preferably 6-acetyl-8-cyclopentyl-5-methyl-2-(5-piperazin-1-yl-pyridin-2-ylamino)-8H-pyrido[2,3-d]pyrimidin-7-one) and an amount of an Aurora inhibitor, wherein the amounts of the cell cycle inhibitor and the Aurora inhibitor when taken as a whole is therapeutically effective for treating said cancer. Aurora kinase inhibitors as used herein refers to inhibitors that produce a direct effect on the signaling pathways that promote growth, proliferation and survival of a cell by inhibiting the function of the Aurora protein. Examples of such inhibitors include those described in International Patent Publications WO 03/106417; WO 03/105855; WO 03/99211; WO 03/79973; and WO 03/31606.

Each of the foregoing patents, patent applications, patent publications and provisional patent applications is herein incorporated by reference in their entirety including all preferences, embodiments, species and subgenera described therein.

The present invention is also directed to certain novel combinations of MEK inhibitors with other STI's, specifically Aurora Kinase inhibitors, PLK inhibitors, CHK1 inhibitors, Raf kinase inhibitors, Akt inhibitors, mTOR inhibitors, bcr-abl tyrosine kinase inhibitors, PDGFR inhibitors, c-Kit inhibitors, erbB inhibitors, VEGF-R inhibitors, FGFR inhibitors and IGF1-R inhibitors.

The present combination invention, (i.e., a combination of a cell cycle inhibitors with other STI's and MEK inhibitors with STI's) further comprises administering one or more additional anti-cancer therapeutic agents selected from the group consisting of alkylating agents, anti-metabolites, antibiotics, hormonal agents, plant-derived antitumor agents, topoisomerase 1/11 inhibitors (such as camptothecin derivatives), antibodies, immunologicals (such as interferons), and/or biological response modifiers.

Alkylating agents include, but are not limited to: AMD-473, altretamine, AP-5280, apaziquone, brostallicin, bendamustine, busulfan, carboquone, carmustine, cyclophosphamide, estramustine, fotemustine, glufosfamide, ifosfamide, KW-2170, mafosfamide, melphalan, mitobronitol, mitolactol, nimustine, nitrogen mustard N-oxide, temozolomide, thiotepa and ranimustine. Platinum-coordinated alkylating compounds include but are not limited to, cisplatin, carboplatin, eptaplatin, lobaplatin, nedaplatin, oxaliplatin or satrplatin.

Antimetabolites include but are not limited to: 5-azacitidine, capecitabine, carmofur, cladribine, clofarabine, cytarabine, decitabine, doxifluridine, eflornithine, enocitabine, ethynylcytidine, 5-fluorouracil (5-FU) alone or in combination with leucovorin, leucovorin, cytosine arabinoside, hydroxyurea, fludarabine, TS-1, gemcitabine, methotrexate, melphalan, 6-mercaptopurine, mercaptopurine, nelarabine, nolatrexed, ocfosfate, disodium premetrexed, pentostatin, pelitrexol, raltitrexed, riboside, tegafur, triapine, trimetrexate, UFT, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid.

Antibiotics include but are not limited to: aclarubicin, actinomycin D, amrubicin, annamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin or zinostatin.

The invention also contemplates the use of the combination of the present invention together with hormonal therapy agents, e.g., exemestane (Aromasin), Lupron, anastrozole (Arimidex), doxercalciferol, fadrozole, formestane, anti-estrogens such as tamoxifen citrate (Nolvadex) and fulvestrant, Trelstar, toremifene, raloxifene, lasofoxifene, letrozole (Femara), or anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl)propionanilide) and combinations thereof.

Plant derived anti-tumor substances include for example those selected from mitotic inhibitors, for example vinblastine, docetaxel (Taxotere) and paclitaxel.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, and topotecan, and combinations thereof.

Immunologicals include interferons and numerous other immune enhancing agents. Interferons include interferon alpha, interferon alpha-2a, interferon, alpha-2b, interferon beta, interferon gamma-1a or interferon gamma-n1. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab, Provenge, Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, or ubenimex.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, TLK-286 or tretinoin.

Non-kinase Anti-angiogenesis agents are another import art active agent that can be combined with the non-kinase combination of the present invention. Anti-angiogenic agents include matrix metalloprotienease inhibitors, such as MMP-2 (matrix-metalloprotienase 2) inhibitors, MMP-9 (matrix-metalloprotienase 9) inhibitors, and COX-II (cyclooxygenase 11) inhibitors, can be used in conjunction with the aforesaid combinations of SDI's in the methods and pharmaceutical compositions described herein. Examples of useful COX-II inhibitors include CELEBREX™ (celecoxib), Bextra (valdecoxib), paracoxib, Vioxx (rofecoxib), and Arcoxia (etoricoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R,3R)1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R,3R)1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;
   and pharmaceutically acceptable salts, solvates and prodrugs of said compounds.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain and Vitaxin.

A combination of the present invention may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Pat. No. 6,682,736 issued Jan. 27, 2004, which is herein incorporated by reference in its entirety.

Further, the invention provides a combination of the present invention with one or more supportive care products, e.g., a product selected from the group consisting of Aloxi, amifostine, ancestim, anethole, aprepitant, BAM-002, CyPat, darbepoetin, dazlizumab, denileukin, dexrazoxane, Emend, etanercept, erythropoietin, Filgrastim (Neupogen), Fragmin, lenograstim, GM-CSF, molgramostim, oprelvekin, ondansetron (Zofran), Procrit, sargramostim, vesnarinone, or combinations thereof. Such conjoint treatment may be achieved by way of the simultaneous, sequential or separate dosing of the individual components of the treatment.

In one embodiment, the additional therapeutic agent is selected from the group consisting of a camptothecin, irinotecan HCl, edotecarin, epirubicin, docetaxel, paclitaxel, exemestane, Lupron, anastrozole, tamoxifen, Trelstar, Filgrastim, ondansetron, Fragmin, Procrit, Aloxi, Emend, and combinations thereof. In a particular embodiment, the additional therapeutic agent is selected from the group consisting of paclitaxel, exemestane, tamoxifen, and combinations thereof.

In a particular embodiment, the invention provides a combination for treating breast cancer comprising a combination of a CDK inhibitor and a MEK inhibitor with a monoclonal antibody (preferably Herceptin), and one or more agents selected from paclitaxel, exemestane, tamoxifen, and combinations thereof.

In a particular embodiment, the invention provides a combination comprising a combination of a CDK inhibitor and a MEK inhibitor (optionally with a monoclonal antibody (preferably Herceptin)), and one or more hormonal agents selected from paclitaxel, exemestane, tamoxifen, and combinations thereof and one or more cytotoxic agents selected from 5-FU, oxaliplatin and leucovorin (or combinations thereof such as prescribed for FOLFOX).

The method of the invention also relates to a method for the treatment of abnormal cell growth in a mammal, including a human, comprising administering to said mammal an amount of a combination of two or more STI's, as defined above, or a pharmaceutically acceptable salt, solvate or prodrug thereof, that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In another aspect the method of the invention is directed to the method of administration of the combination. More particularly the active agents of the combination therapy are administered sequentially in either order or simultaneously. When the active agents are administered simultaneously, one skilled in the art will understand that the second agent can be administered some time after the first agent. The particular period of delay is dependent on the particular pharmacokinetic and formulation parameters of the active agent.

In another aspect of the invention is the minimization of the combination dose. It is frequently the case that the individual dosage regimes for the active agents can lead to undesirable side effects that can potentially lead to a discontinuation of the medication. One particular preferred embodiment of the invention is to reduce the dosage to the minimum dose necessary to treat the cancer. Thus one preferred embodiment is the administration of a combination wherein the amounts of both active agents is less than the efficacious dose of either agent alone. Another embodiment of the invention is the administration of a combination that has activity above the activity of either agent alone. Preferred combinations are those in which the combination is synergistic compared to either alone. Preferably, the combination is superadditive.

This invention also relates to a kit for treatment of abnormal cell growth, comprising a combination as defined above, and written instructions for administration of all components. In a particular aspect the specific CDK inhibitor and its method of administration is described in the written instructions. In another particular aspect of the kit of the invention, the written instructions specify the MEK inhibitor and describe its method of administration. In one embodiment of said kit, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said kit, said abnormal cell growth is a benign proliferative disease, including, but not limited to, psoriasis, benign prostatic hypertrophy or restenosis.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

Those active compounds of the present combination invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the active compounds of the present combination invention can be substituted for bioisosteric groups, that is, groups that have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of the combinations of the present invention may also exist as tautomers. This invention relates to the use of all such tautomers and mixtures thereof.

The subject matter of the invention also includes isotopically-labelled compounds, and the pharmaceutically acceptable salts, solvates and prodrugs thereof, which are identical to those recited for the active compounds described herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled active compounds of the combinations of this invention and prodrugs thereof can generally be prepared by procedures well known to those skilled in the art.

This invention also encompasses pharmaceutical compositions containing and methods of treating cancer through administering prodrugs of the active compounds of the present combination invention. Active compounds having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of the active compounds. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The terms synergy and synergistic mean that the combination of two or more effectors or active agents is at least greater than the activity of either agent alone and is preferably at least additive in their effect. More preferably, the synergy is greater than additive. Most preferably, the synergy is superadditive. The term “additive” is use to mean that the result of the combination of the two or more effectors or agents is more than the sum of each effector or agent together and preferably at least 10 percent greater than the combination's additive effect. The term “superadditive” is used to mean that the result of combination of two or more effectors is at least 25 percent greater than the combination's additive effect.

### DETAILED DESCRIPTION OF THE INVENTION

Definitions and Abbreviations

Unless otherwise indicated, this disclosure uses definitions provided below.

The term “cancer” includes, but is not limited to, the following cancers: cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's disease, hairy cells, and leukemia.

The phrase “pharmaceutically acceptable” refers to substances, which are within the scope of sound medical judgment, suitable for use in contact with the tissues of patients without undue toxicity, irritation, allergic response, and the like, commensurate with a reasonable benefit/risk ratio, and effective for their intended use.

“Ligand” is particularly used to describe a small molecule that binds to a receptor. An important class of ligands in the instant invention are those antibodies described herein above which bind to receptors in the epidermal growth factor family. Ligands can be inhibitors of receptor function and can be antagonists of the action of activators.

Certain abbreviations common in the art are freely used and will be understood in context. Among these are pharmacokinetics (PK), pharmacodynamics (PD), fetal bovine serum (FBS), penicillin/streptomycin (pen/strep), Roswell Park Memorial Institute (RPMI), per os (PO), once per day (QD), interaperitoneally (IP), subcutaneously (SC), enzyme-linked immunosorbent assay (ELISA), the maximum concentration of an analyte in a PK analysis (Cₘₐₓ), and the average concentration of an analyte in a PK analysis (Cₐᵥₑ).

The term “treating” refers to reversing, alleviating, inhibiting the progress of, or preventing a disorder or condition to which such term applies, or to preventing one or more symptoms of such disorder or condition.

The term “treatment” refers to the act of “treating,” as defined immediately above.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “as a whole is therapeutically effective” refers to the total dose of the combination agents that produces the therapeutic effect. One skilled in the art will appreciate that the present invention envisions timing the application of the amount of the individual agents simultaneous, sequential or via a separate dosing schedule. Thus, the physician will target a therapeutic effect, e.g. a reduction in tumor size, based on the collective amounts of the components independent of the ordering or the time lag between administration.

TABLE 1
Abbreviations

| Abbreviation | Description |
|---|---|
| aq | Aqueous |
| ACN | Acetonitrile |
| BOC | tert-butoxycarbonyl |
| DCM | Dichloromethane |
| DSC | differential scanning calorimetry |
| Et₃N | Triethylamine |
| EtOH | ethyl alcohol |
| h, min, s | hour, minute, second |
| IPA | isopropyl alcohol |
| MeOH | Methanol |
| PXRD | powder X-ray diffraction |
| RH | relative humidity |
| RT | room temperature, i.e. 20° C.-25° C. |
| THF | Tetrahydrofuran |
| mgA/mL | milligrams of active substance per milliliter of solution |

Cyclin dependent kinase inhibitors as used herein refers to inhibitors that produce a direct effect on the signaling pathways that promote growth, proliferation and survival of a cell by inhibiting the activity of any of the known cyclin dependant kinase, most preferably CDK4/6. CDK inhibitors can be prepared by methods known to those skilled in the art. Preferred CDK4/6 inhibitors can be prepared by the methods described in International Publication WO 03/062236, published Jul. 31, 2003 and U.S. Patent Application 60/486,351 filed Jul. 11, 2003 and 60/440,805 filed Jan. 17, 2003. Other specific methods of preparation of CDK's are described in EP1250353, WO 02/96888, WO 03/076437, WO 03/76436, WO 03/76434 WO 01/64368, U.S. Provisional Application No. 60/491,474 and U.S. Provisional Application No. 60/491,474.

Signal transduction inhibitors (STI) as used herein refers to inhibitors that produce a direct effect on the signaling pathways that promote growth, proliferation and survival of a cell. One skilled in the art will appreciate that STI's so defined includes Growth Factors and mitogen stimulated kinase signal transduction pathway inhibitors. Such inhibitors also include small molecules, antibodies, and antisense molecules. Examples of such signal transduction inhibitors include tyrosine kinase inhibitors, serine/threonine kinase inhibitors, dual specificity kinase inhibitors, lipid kinase inhibitors, histone deacetylase inhibitors, Bc12 inhibitors, p53 inhibitors, MDMZ inhibitors, Ras inhibitors and Hsp 90 inhibitors.

Methods for preparing such agents are well known in the literature and to those skilled in the art.

Dual specificity kinase inhibitors include MEK inhibitors.

MEK inhibitors as used herein refers to inhibitors that produce a direct effect on the signaling pathways that promote growth, proliferation and survival of a cell by inhibiting the activity of MEK1 or MEK2. Examples of such most preferred inhibitors include 2-(2-chloro-4-iodo-phenylamino)-N-cyclopropylmethoxy-3,4-difluoro-benzamide and N-[(R)-2,3-Dihydroxy-propoxy]-3,4-difluoro-2-(2-fluoro-4-iodo-phenylamino)-benzamide. 2-(2-chloro-4-iodo-phenylamino)-N-cyclopropylmethoxy-3,4-difluoro-benzamide and N-[(R)-2,3-Dihydroxy-propoxy]-3,4-difluoro-2-(2-fluoro-4-iodo-phenylamino)-benzamide are selective MEK 1 and MEK 2 inhibitors. Selective MEK 1 or MEK 2 inhibitors are those compounds which inhibit the MEK 1 or MEK 2 enzymes without substantially inhibiting other enzymes such as MKK3, ERK, PKC, Cdk2A, phosphorylase kinase, EGF and PDGF receptor kinases, and C-src. In general, a selective MEK 1 or MEK 2 inhibitor has an IC₅₀ for MEK 1 or MEK 2 that is at least one-fiftieth (1/50) that of its IC₅₀ for one of the above-named other enzymes. A selective inhibitor may have an IC₅₀ that is at least 1/100, 1/500, or even 1/1000, 1/5000 or less than that of its IC₅₀ for one or more of the above-named enzymes.

A compound which is a MEK inhibitor may be determined by using an assay known to one of skill in the art that measures MEK inhibition. For example, MEK inhibition may be determined using the assays titled, “Enzyme Assays” in U.S. Pat. No. 5,525,625, column 6, beginning at line 35. The complete disclosure of U.S. Pat. No. 5,525,625 is hereby incorporated by reference. Specifically, a compound is an MEK inhibitor if a compound shows activity in the assay titled, “Cascade Assay for Inhibitors of the MAP Kinase Pathway,” column 6, line 36 to column 7, line 4 of the U.S. Pat. No. 5,525,625 and/or shows activity in the assay titled, “In Vitro MEK Assay” at column 7, lines 4 to 27 of the above-referenced patent. Alternatively, MEK inhibition may be measured in the assay described in WO 02/06213 A1, the complete disclosure of which is hereby incorporated by reference.

Examples of methods of preparing MEK inhibitors according to the present invention include, but are not limited to the methods disclosed in the following PCT Publications: WO 99/01426, WO 99/01421, WO 00/42002, WO 00/42022, WO 00/41994, WO 00/42029, WO 00/41505, WO 00/42003, WO 01/68619, and WO 02/06213.

Another MEK inhibitor embodiment of the invention includes the compound 1,4-diamino-2,3-dicyano-1,4-bis[2-aminophenylthio]butadiene (U-0126) which can be prepared by methods well known to those skilled in the art.

Serine/threonine kinase inhibitors include Raf kinase inhibitors, Akt inhibitors and mTOR inhibitors.

Raf kinase inhibitors may be prepared by those methods described in WO 03/68223, published Aug. 21, 2003, WO 03/82272, published Oct. 9, 2003, WO 03/22840, published Mar. 20, 2003, WO 03/22838, published Mar. 20, 2003, WO 03/22837, published Mar. 20, 2003, WO 03/22836, published Mar. 20, 2003, and WO 03/22833, published Mar. 20, 2003.

Akt inhibitors may be prepared according to the methods described in European Patent Publication EP 1379251, and International Publications WO 03/86403, WO 03/86394 and WO03/86279, all published Oct. 23, 2003.

mTOR inhibitors may be prepared according to the methods described in European Patent 648,494, International Patent Publications WO 03/64383, WO 96/41865, WO 99/36533, WO 01/14387; and U.S. Pat. Nos. 5,525,610, 5,310,903, 5,362,718 and 5,527,907. Examples of such inhibitors include rapamycins, preferably rapamycin, CCI 779, Rad001 and Arry 142886.

Tyrosine kinase STI inhibitors include but are not limited to bcr-abl tyrosine kinase inhibitors, PDGFR inhibitors, c-Kit inhibitors, erbB inhibitors, VEGF-R inhibitors, FGFR inhibitors and IGF1-R inhibitors.

Bcr-abl tyrosine kinase inhibitors can be prepared by methods well known to those skilled in the art. Specific methods are described in European Patent Publication 564,409, issued Jan. 19, 2000. Examples of such inhibitors include Gleevec.

PDGFR inhibitors may be prepared by methods well known to those skilled in the art. Specific methods are described in U.S. patent application Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999). Other methods are described in U.S. Provisional Patent Applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000).

c-Kit inhibitors may be prepared by methods well known to those skilled in the art. Specific methods are described in International Patent Publications WO 03/028711, published Apr. 10, 2003 and WO 03/002114, published Jan. 9, 2003.

ErbB inhibitors include erbB1 and/or erbB2 inhibitors. Numerous such compounds are under clinical trials currently and methods for their preparation are well know to those skilled in the art.

EGFR (Erbb1) inhibitors and their methods of preparation are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

ErbB2 inhibitors may be prepared by those methods described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors and methods for their preparation are also described in U.S. Provisional Application No. 60/117,341, filed Jan. 27, 1999, and in U.S. Provisional Application No. 60/117,346, filed Jan. 27, 1999, both of which are herein incorporated by reference in their entirety. Other erbb2 receptor inhibitors include TAK-165 (Takeda) and GW-572016 (Glaxo-Wellcome). Pan-erBB inhibitors (active against erbB1 and erb2) and methods for their preparation are described in U.S. Pat. No. 5,464,861 issued Nov. 17, 1995, U.S. Pat. No. 5,654,307 issued Aug. 5, 1997, U.S. Pat. No. 6,344,459 issued Feb. 5, 2002 U.S. Pat. No. 6,127,374 issued Oct. 3, 2000, U.S. Pat. No. 6,153,617 issued Nov. 28, 2000, U.S. Pat. No. 6,344,455 issued Feb. 5, 2002, U.S. Pat. No. 6,664,390 issued Dec. 16, 2003 and International Publication WO 02/00630 published Jan. 3, 2002.

ErbB2 receptor inhibitors include GW-282974 (Glaxo Wellcome plc), the monoclonal antibodies AR-209 (Aronex Pharmaceuticals Inc. of The Woodlands, Tex., USA) and 2B-1 (Chiron), Herceptin, 2C4, and pertuzumab.

Certain other erbB2 inhibitors useful in the treatment of cancer are disclosed in WO 01/98277, the disclosure of which is incorporated herein in its entirety. Various other compounds, such as styrene derivatives, have also been shown to possess tyrosine kinase inhibitory properties. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to methods of preparing certain bicyclic derivatives, in particular quinazoline derivatives. Also, World Patent Application WO 92/20642 (published Nov. 26, 1992), refers to methods for preparing certain bis-mono and bicyclic aryl and heteroaryl tyrosine kinase inhibiting compounds and states that they are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are U.S. patent application Ser. No. 09/488,350 (filed Jan. 20, 2000) and Ser. No. 09/488,378 (filed Jan. 20, 2000), both of which are incorporated herein by reference in their entirety.

General synthetic methods which may be referred to for preparing the erbB2 compounds of the present invention are provided in U.S. Pat. No. 5,747,498 (issued May 5, 1998), U.S. patent application Ser. No. 08/953,078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30, 1997), WO 95/19774 (published Jul. 27, 1995) and WO 97/13771 (published Apr. 17, 1997). Additional procedures are referred to in U.S. patent application Ser. No. 09/488,350 (filed Jan. 20, 2000) and Ser. No. 09/488,378 (filed Jan. 20, 2000). The foregoing patents and patent applications are incorporated herein by reference in their entirety. Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M., Kazmierczak, F., Leonard, N.J., J. Org. Chem. 1986, 51, 5, p. 616. Palladium-catalyzed boronic acid couplings are described in Miyaura, N., Yanagi, T., Suzuki, A. Syn. Comm. 1981, 11, 7, p. 513. Palladium catalyzed Heck couplings are described in Heck et. al. Organic Reactions, 1982, 27, 345 or Cabri et. al. in Acc. Chem. Res. 1995, 28, 2. For examples of the palladium catalyzed coupling of terminal alkynes to aryl halides see: Castro et. al. J. Org. Chem. 1963, 28, 3136. or Sonogashira et. al. Synthesis, 1977, 777. Terminal alkyne synthesis may be performed using appropriately substituted/protected aldehydes as described in: Colvin, E. W. J. et. al. Chem. Soc. Perkin Trans. I, 1977, 869; Gilbert, J. C. et. al. J. Org. Chem., 47, 10, 1982; Hauske, J. R. et. al. Tet. Lett., 33, 26, 1992, 3715; Ohira, S. et. al. J. Chem. Soc. Chem. Commun., 9, 1992, 721; Trost, B. M. J. Amer. Chem. Soc., 119, 4, 1997, 698; or Marshall, J. A. et. al. J. Org. Chem., 62, 13, 1997, 4313.

Alternatively terminal alkynes may be prepared by a twostep procedure. First, the addition of the lithium anion of TMS (trimethylsilyl) acetylene to an appropriately substituted/protected aldehyde as in: Nakatani, K. et. al. Tetrahedron, 49, 9, 1993, 1901. Subsequent deprotection by base may then be used to isolate the intermediate terminal alkyne as in Malacria, M.; Tetrahedron, 33, 1977, 2813; or White, J. D. et. al. Tet. Lett., 31, 1, 1990, 59.

Starting materials, the synthesis of which is not specifically described above, are either commercially available or can be prepared using methods well known to those of skill in the art.

Antibodies to erbB2 are known and have therapeutic utility. U.S. Pat. No. 5,725,856 is directed, in part, to treatment by administering an antibody that binds to the extracellular domain of the erbB2 (HER2) receptor. U.S. Pat. No. 5,677,171 is directed to a monoclonal antibody that binds the HER2 receptor. U.S. Pat. No. 5,720,954 is directed to a treatment by use of a cytotoxic factor and an antibody to HER2 receptor. U.S. Pat. No. 5,770,195 is directed to inhibiting the growth of tumor cells. U.S. Pat. No. 6,165,464 is directed to an isolated human antibody that binds HER2 receptor. U.S. Pat. No. 6,387,371 is directed to a method of treating a cancer by administering an antibody and a factor which suppresses cancer cell growth.

The erbB gene can be erbB1, erbB2, erbB3, erbB4, or combinations thereof. In one aspect, the gene is erbB1. In another aspect, the gene is erbB2. In yet another aspect, the gene is erbB3. In still another aspect, the gene is erbB4.

In one aspect of the invention, the antibody can recognize the extracellular domain of the protein.

VEGF inhibitors may be prepared by methods described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. WO 01/02369 (published Jan. 11, 2001); U.S. Provisional Application No. 60/491,771 (filed Jul. 31, 2003); U.S. Provisional Application No. 60/460,695 (filed Apr. 3, 2003); and WO 03/106462A1 (published Dec. 24, 2003). Other examples of methods of preparing VEGF inhibitors are described in International Patent Publications WO 99/62890 published Dec. 9, 1999, WO 01/95353 published Dec. 13, 2001 and WO 02/44158 published Jun. 6, 2002. Methods of preparing other examples of some specific VEGF inhibitors (including IM862 (Cytran Inc. of Kirkland, Wash., USA); Avastin, an anti-VEGF monoclonal antibody of Genentech, Inc. of South San Francisco, Calif.; and angiozyme, a synthetic ribozyme from Ribozyme (Boulder, Colo.) and Chiron (Emeryville, Calif.)) are well known to those skilled in the art.

Signal Transduction Inhibitors comprise a broad population of inhibitors. Most of these inhibitors fall are either tyrosine kinase inhibitors or serine/threonine kinase inhibitors. However, there are a small number of STI's that are not tyrosine kinase inhibitors or serine/threonine kinase inhibitors. Examples of such inhibitors include dual specificity kinase inhibitors, lipid kinase inhibitors, histone deacetylase inhibitors, Bc12 inhibitors, p53 inhibitors, MDMZ inhibitors, Ras inhibitors, Hsp 90 inhibitors, K-Ras inhibitors and TGFbeta-R inhibitors. Such inhibitors can be prepared by methods well known to those skilled in the art.

PI3 kinase inhibitors may be prepared by methods well known to those skilled in the art. Specific methods are described in WO 01/81346; WO 01/53266; and WO 01/83456. U.S. patent application Ser. No. 10/730,680 (filed Dec. 8, 2003); U.S. patent application Ser. No. 10/743,852 (filed Dec. 22, 2003); U.S. Provisional Patent Application No. 60/475,970 (filed Jun. 5, 2003); U.S. Provisional Patent Application No. 60/475,992 (filed Jun. 5, 2003); U.S. Provisional Patent Application No. 60/476,073 (filed Jun. 5, 2003); U.S. Provisional Patent Application No. 60/476,251 (filed Jun. 5, 2003); U.S. Provisional Patent Application No. 60/475,971 (filed Jun. 5, 2003); and U.S. Provisional Patent Application No. 60/476,057 (filed Jun. 5, 2003). The method of the invention comprises treating a mammal having a cancer, comprising: administering to said mammal in need of such treatment, sequentially in either order, simultaneously, or both, (i) a therapeutically effective amount of a first STI compound, as defined above, and (ii) a therapeutically effective amount of a second STI compound, as defined above. In a preferred embodiment, the method of the invention comprises treating a human having a cancer, comprising: administering to said human in need of such treatment, sequentially in either order, simultaneously, or both, (i) a therapeutically effective amount of a first STI compound, as defined above, and (ii) a therapeutically effective amount of a second STI compound, as defined above.

The cancer can be a solid cancer. In a particular aspect the cancer is not a solid tumor, including, for example, a leukemia or a lymphoma. The volume of the solid cancer can decrease upon administration of the method of the invention.

When the STI is an antibody it can be either a polyclonal or monoclonal antibody. In a particular aspect, the antibody is a monoclonal antibody. Thus, the antibody can be selected from the group consisting of Herceptin, 2C4, and pertuzumab. In one embodiment the antibody is pertuzumab. In another embodiment, the antibody is 2C4. In yet another embodiment, the antibody is Herceptin.

The amount of Herceptin administered can be less than about 2 mg/kg/week. In one aspect, the amount of Herceptin administered is about 0.6 mg/kg/week. The antibody can be administered at least about once per week. In another aspect, the antibody can be administered about once per two weeks.

The method of the invention may be useful with a cancer characterized by amplification of a specific STI gene, an overexpression of the STI protein, or both. In one aspect, the STI gene, the STI protein, or both, are CDK4/6. The overexpression can be characterized by a +2 or +3 level. Any method standard in the art can used to measure the levels of amplification or overexpression. For example, the amplification can be measured by fluorescence in situ hybridization (FISH). An advantageous method is described by Coussens et al. Science 230, 1132 (1032). Overexpression can be measured by immunohistochemistry (IHC). An advantageous method is also described by Coussens et al. Id. Alternatively, the level of overexpression of STI is inferred from clinical observations, without use of explicit measurement by IHC, but based rather on the patient history, the physical diagnosis or other elements of the diagnosis.

The antibody can advantageously be a mediator of antibody-dependent cellular cytotoxicity.

In one aspect of the method of the invention, the combination is administered at least about daily. In another aspect, the combination of the invention is administered at least about twice daily. The therapeutically effective amount of the first compound can be about 25 mg/kg/day. In another aspect, the therapeutically effective amount of the first compound is about 50 mg/kg/day.

The combination of the invention can be administered orally, buccally, sublingually, vaginally, intraduodenally, parenterally, topically, or rectally. The formulation will preferably be adapted to the particular mode of administration. Antibody combinations of the invention can be administered substantially simultaneously with the other compounds of the combination. The formulations of the individual components of the combination is dependent on the properties of each agent and the desired pharmacological effect desired by the administrator.

The method of the invention is applicable to a human. Non-humans can also be treated. For example, the mammal can be a horse.

The method of the invention is useful for administration to female mammals. The method can also be useful for males. The mammal can be an adult. In another aspect, infants, children, adolescents or the elderly can be treated with the methods of the invention.

The methods of the invention are applicable to a wide variety of abnormal cell growth conditions. In one aspect, the methods and kits are advantageously applied to cancers. The cancer can be selected from the group consisting of: lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. Other cancers can also be susceptible to treatment with the methods of the invention. In one aspect, the cancer is selected from the group consisting of ovarian cancer and breast cancer. In another aspect, the cancer is breast cancer.

The method of the invention is also applicable to adjuvant therapy, for example, in which the mammal, has received or is receiving a course of chemotherapeutic agents. In such an aspect, the remaining cancer may be a minimal residual disease. In another aspect, the method of the invention can be applied as a prophylactic measure. Thus, for example, the method can be applied to a mammal in cancer remission, in which no measurable disease can be detected.

In one aspect of the methods of the invention, the amount of the active agents is at least sufficient to produce therapeutic synergy. In consequence, the combination of the steps of the method of the invention is an improved treatment of a cancer when compared to either alone.

The invention also comprises a kit comprising: (a) a first agent, as described above, and (b) written instructions packaged with (a), for simultaneous or sequential administration for the treatment of a cancer. Thus, the written instructions can elaborate and qualify the modes of administration.

In one aspect of the kit, the written instructions specify administration of a cyclin dependent kinase inhibitor and a STI. Advantageously, the written instructions specify administration of 6-acetyl-8-cyclopentyl-5-methyl-2-(5-piperazin-1-yl-pyridin-2-ylamino)-8H-pyrido[2,3-d]pyrimidin-7-one. In another particular aspect of the kit, the kit further comprises administration of 2-(2-chloro-4-iodo-phenylamino)-N-cyclopropylmethoxy-3,4-difluoro-benzamide or N-[(R)-2,3-Dihydroxy-propoxy]-3,4-difluoro-2-(2-fluoro-4-iodo-phenylamino)-benzamide. Moreover, the kit can comprise a fluid for reconstituting the aforesaid active agents, if supplied in the dry state.

The compounds of the present combination invention are potent ST inhibitors of oncogenic and protooncogenic protein tyrosine kinases and thus are all adapted to therapeutic use as antiproliferative agents (e.g., anticancer) in mammals, particularly in humans. In particular, the compounds of the present invention are useful in the prevention and treatment of a variety of human hyperproliferative disorders such as malignant and benign tumors of the kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that the methods and kits of the present invention may be effective against a range of leukemias and lymphoid malignancies.

The compounds of the present combination invention may also be useful in the treatment of additional disorders in which aberrant expression ligand/receptor interactions or activation or signaling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signaling of the specific STI involved. In addition, the active compounds of the present invention may have therapeutic utility in inflammatory, angiogenic and immunologic disorders involving both identified and as yet unidentified tyrosine kinases that are inhibited by the compounds of the present invention.

The disclosed active compounds embrace all pharmaceutically acceptable isotopic variations. An isotopic variation is a compound in which at least one atom is replaced by an atom having the same atomic number, but an atomic mass different from the atomic mass usually found in nature. Useful isotopes include isotopes of hydrogen, carbon, nitrogen, oxygen, phosphorus, sulfur, fluorine, and chlorine. Exemplary isotopes thus include, without limitation, ²H, ³H, ¹³C, ¹⁴C, ¹⁵N, ¹⁷O, ¹⁸O, ³²P, ³⁵S, ¹⁸F, and ³⁶Cl.

Substitution of the disclosed compounds with isotopes such as deuterium, i.e. ²H, may afford certain therapeutic advantages resulting from greater metabolic stability, for example, increased in vivo half-life or reduced dosage requirements, and hence may be more useful in some circumstances. In addition, certain isotopic variations, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variations of the disclosed compounds can generally be prepared by conventional techniques known to those skilled in the art or by processes analogous to those described in the accompanying Examples using appropriate isotopic variations of suitable reagents. Pharmaceutically acceptable solvates of the disclosed compounds include those in which the solvent of crystallization may be isotopically substituted, e.g. D₂O, dr-acetone, d₆-DMSO.

The disclosed active compounds may be administered as crystalline or amorphous products. They may be obtained, for example, as solid plugs, powders, or films by methods such as precipitation, crystallization, freeze-drying, spray drying, or evaporative drying. Microwave or radio frequency drying may be used for this purpose.

The disclosed combinations may be administered alone or in combination with other drugs and will generally be administered as a formulation in association with one or more pharmaceutically acceptable excipients. The term “excipient” describes any ingredient other than the active agents described herein and their salts. The choice of excipient will to a large extent depend on the particular mode of administration.

The disclosed compounds may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations. Liquid formulations include suspensions, solutions, syrups and elixirs. Such formulations may be employed as fillers in soft or hard capsules and typically comprise a carrier, for example, water, EtOH, polyethylene glycol, propylene glycol, methylcellulose, or a suitable oil, and one or more emulsifying agents and/or suspending agents. Liquid formulations may also be prepared by the reconstitution of a solid, for example, from a sachet.

The disclosed compounds may also be used in fast-dissolving, fast-disintegrating dosage forms such as those described in Liang and Chen, Expert Opinion in Therapeutic Patents (2001) 11(6):981-986.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methylcellulose, microcrystalline cellulose, lower alkyl-substituted hydroxypropyl cellulose, starch, pregelatinized starch, and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose, and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch, and dibasic calcium phosphate dihydrate.

Tablets may also optionally include surface-active agents, such as sodium lauryl sulfate and polysorbate 80, and glidants such as silicon dioxide and talc. When present, surface-active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulfate. Lubricants generally comprise from 0.25 wt % to 10 wt %, preferably from 0.5 wt % to 3 wt % of the tablet. Other ingredients may include preservatives, anti-oxidants, flavors, and colorants.

Tablet blends may be directly compressed to form tablets. Tablet blends or portions of blends may alternatively be wet-, dry-, or melt-granulated, melt congealed, or extruded before tabletting. The final formulation may comprise one or more layers and may be coated or uncoated. Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant. For additional details concerning the formulation of tablets, see H. Lieberman and L. Lachman, Pharmaceutical Dosage Forms: Tablets, Vol. 1 (1980).

Solid formulations for oral administration may be formulated to be immediate and/or modified release. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14. For a discussion of the use of chewing gum to achieve controlled release, see WO 00/35298.

The disclosed compounds may also be administered directly into the blood stream, into muscle, or into an internal organ. Suitable means for parenteral administration include intravenous, intra-arterial, intraperitoneal, intrathecal, intraventricular, intraurethral, intrasternal, intracranial, intramuscular, and subcutaneous. Suitable devices for parenteral administration include needle (including micro-needle) injectors, needle-free injectors and infusion techniques.

Parenteral formulations are typically aqueous solutions which may contain excipients such as salts, carbohydrates, and buffering agents (preferably to a pH of from 3 to 9), but for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art.

The solubility of the disclosed compounds used in the preparation of parenteral solutions may be increased by the use of appropriate formulation techniques, such as the incorporation of solubility-enhancing agents. Formulations for parenteral administration may be formulated to be immediate and/or modified release as described above. Thus the disclosed compounds may be formulated in a more solid form for administration as an implanted depot providing long-term release of the active compound.

The compounds of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibers, bandages, and microemulsions. Liposomes may also be used. Typical carriers include alcohol, water, mineral oil, liquid petrolatum, white petrolatum, glycerin, polyethylene glycol and propylene glycol. Topical formulations may also include penetration enhancers. See, for example, Finnin and Morgan, J Pharm Sci (1999) 88(10):955-958.

Other means of topical administration include delivery by iontophoresis, electroporation, phonophoresis, sonophoresis and needle-free (e.g. POWDERJECT) or micro-needle injection. Formulations for topical administration may be formulated to be immediate and/or modified release as described above.

The disclosed compounds can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurized container, pump, spray, atomizer (preferably an atomizer using electrohydrodynamics to produce a fine mist), or nebulizer, with or without the use of a suitable propellant, such as dichlorofluoromethane. The pressurized container, pump, spray, atomizer, or nebulizer contains a solution or suspension, which comprises the active compound, an agent for dispersing, solubilizing, or extending release of the active compound (e.g., EtOH or aqueous EtOH), one or more solvents, which serve as a propellant, and an optional surfactant, such as sorbitan trioleate or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules, blisters and cartridges (made, for example, from gelatin or hydroxypropylmethyl cellulose) for use in an inhaler or insufflator may be formulated to contain a powder mix of the active compound, a suitable powder base such as lactose or starch, and a performance modifier such as L-leucine, mannitol, or magnesium stearate. The lactose may be anhydrous or, preferably, monohydrated. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the compound of the invention per actuation and the actuation volume may vary from 1 μl to 100 μl. A typical formulation may comprise a compound of Formula 1 or Formula 2, propylene glycol, sterile water, EtOH, and NaCl. Alternative solvents, which may be used instead of propylene glycol, include glycerol and polyethylene glycol.

Formulations for inhaled/intranasal administration may be formulated to be immediate and/or modified release using, for example, poly(DL-lactic-coglycolic acid (PGLA). Suitable flavors, such as menthol and levomenthol, or sweeteners, such as saccharin or saccharin sodium, may be added to formulations intended for inhaled/intranasal administration.

In the case of dry powder inhalers and aerosols, the dosage unit is determined by means of a valve that delivers a metered amount. Units in accordance with the invention are typically arranged to administer a metered dose or “puff” containing from 100 to 1000 μg of the active pharmaceutical ingredient. The overall daily dose will typically be in the range 100 μg to 10 mg which may be administered in a single dose or, more usually, as divided doses throughout the day.

The active compounds may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release as described above.

The disclosed compounds may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer (e.g., hydroxypropylmethylcellulose, hydroxyethylcellulose, or methyl cellulose), or a heteropolysaccharide polymer (e.g., gelan gum), may be incorporated together with a preservative, such as benzalkonium chloride. Such formulations may also be delivered by iontophoresis. Formulations for ocular/andial administration may be formulated to be immediate and/or modified release as described above.

The disclosed compounds may be combined with soluble macromolecular entities such as cyclodextrin or polyethylene glycol-containing polymers to improve their solubility, dissolution rate, taste masking, bioavailability and/or stability. Drug-cyclodextrin complexes, for example, are found to be generally useful for most dosage forms and administration routes. Both inclusion and non-inclusion-complexes may be used. As an alternative to direct complexation with the drug, the cyclodextrin may be used as an auxiliary additive, i.e. as a carrier, diluent, or solubilizer. Alpha-, beta- and gamma-cyclodextrins are commonly used for these purposes. See, for example, International Patent Applications WO 91/11172, WO 94/02518, and WO 98/55148.

The dose of the individual compounds of the present combo invention will vary from approximately 0.01 mg/kg to approximately 100 mg/kg of body weight per day. Typical adult doses will be approximately 0.1 mg to approximately 3000 mg per day. The quantity of active component in a unit dose preparation may be varied or adjusted from approximately 0.1 mg to approximately 500 mg, preferably from about 0.6 mg to 100 mg according to the particular application and the potency of the active component. The composition can, if desired, also contain other compatible therapeutic agents. A subject in need of treatment is administered a dosage of about 0.6 to about 500 mg per day, either singly or in multiple doses over a 24-hour period. Such treatment may be repeated at successive intervals for as long as necessary.

The following protocols demonstrate the efficacy of the combinations of the invention.

### Chemotherapeutic Evaluation of a CDK-4 Inhibitor in Combination with the Standard Agent Taxotere, Against Advanced Stage Human Breast Carcinoma MDA-MB-435

Objective: Determine the in vivo efficacy of a CDK-4 inhibitor and the standard agent Taxotere, when administered, as both single agents and together, in two combination schedules, against the solid human xenograft MDA-MB-435.

Methods: Four hundred female SCID mice were received from Charles River Breeding Laboratories on Feb. 11, 2003. Mice were 21-28 days old upon arrival. All animals were examined prior to the initiation of the study to ensure that they were healthy and acclimated to the laboratory environment. Mice were housed in barrier facilities with food and water provided ad libitum on a 12-hour light/dark cycle. Animals housed 5 per cage while in the tumor pool, and 4 per cage when randomized into study groups. Animal care was provided in accordance with AAALAC guidelines. All protocols involving animals were reviewed and approved by the institutional animal care and use committee.

On day 0 (Feb. 20, 2003) all mice (18-22 g) received a subcutaneous (SC) implant of MDA-MB-435 tumor fragment, approximately 30 mg, from tumors weighing 1000 mg. The solid human tumor model MBA-MB-435, a breast carcinoma, was developed from cell lines and maintained in SCID mice. The tumor model was serially passed as SC implants of tumor fragments. Tumor source for this study was MDA-MB-435/14 (T, 1-29-03).

Treatment was initiated when tumor fragments reached 200-250 mg in size. Animals were randomized and then sorted into twenty-four groups of 8 mice. Animals were individually weighed and tumors measured the day treatment was initiated, and every 3-4 days during treatment. Dosing solutions were calculated according to average group weight. 6-Acetyl-8-cyclopentyl-5-methyl-2-(5-piperazin-1-yl-pyridin-2-ylamino)-8H-pyrido[2,3-d]pyrimidin-7-one and Taxotere, were prepared according to the drug preparation sheets, and as noted on the chemotherapy drug preparation data sheet. The compounds were administered as single agents, and in two different schedules. One set of animals was dosed with 6-acetyl-8-cyclopentyl-5-methyl-2-(5-piperazin-1-yl-pyridin-2-ylamino)-8H-pyrido[2,3-d]pyrimidin-7-one first for 5 days, followed in 24 hours by the standard agent (Taxotere) for one IV dose. The second set was dosed with the standard agent (Taxotere) for one dose, followed in 24 hours by 5 daily doses of 6-acetyl-8-cyclopentyl-5-methyl-2-(5-piperazin-1-yl-pyridin-2-ylamino)-8′-pyrido[2,3-d]pyrimidin-7-one. These schedules were repeated three times. The solution of 6-acetyl-8-cyclopentyl-5-methyl-2-(5-piperazin-1-yl-pyridin-2-ylamino)-8H-pyrido[2,3-d]pyrimidin-7-one was administered by oral gavage (PO) once daily, on days 14-18, 21-25, and 28-32, or days 15-19, 22-26, and 29-33, according to the treatment schedule. Taxotere was administered IV on days 19, 26, and 33, or days 14, 21, and 28, according to the treatment schedule. An additional group of 16 mice were included as negative control and were administered both dosing vehicles (50 mM sodium lactate for 6-acetyl-8-cyclopentyl-5-methyl-2-(5-piperazin-1-yl-pyridin-2-ylamino)-8H-pyrido[2,3-d]pyrimidin-7-one, pH 4.0, and Tween 80/EtOH/Water for Taxotere) PO on days 14-18, 21-25, and 28-32, and IV on days 19, 26, and 33. The PO dosing volume was 0.5 ml, the IV dosing volume was 0.2 ml.

During the course of the study the animals were weighed to calculate treatment doses and weekly thereafter. Tumors were measured every three to four days throughout the study. All animals were observed for clinical signs daily and after compound administration. All data collection was on CRAAS, and all printed reports were appropriately documented in the study file (databook 83410×13). This study was conducted in accordance with established departmental SOP's.

TABLE 1
CCTM Protocol Summary
Expt. No.: 83410x13 Tumor: MDA-MB-435 Mouse: CB17 SCID Sex: F

| Cage | No. | Tumor | RX | | Dose (mg/kg/inj) | | Schedule | | Route | |
|---|---|---|---|---|---|---|---|---|---|---|
| No. | of Mice | Inoculum | Test | Standard | Test | Standard | Test | Standard | Test | Std. |
| 1 | 16 | +SC | Lactate | Tw/EtOH/Water | 0.5 mL | 0.2 mL | Q1Dx5,2D | 24 Hr post 5^{th} PO | PO | IV |
| | | | | | | | off. X3 | dose X3 | | |
| | | | | | | | @200-250 mg | @200-250 mg | | |
| 2 | 8 | +SC | 0332991 | — | 210 | — | Q1Dx5,2D | — | PO | — |
| | | | | | | | off. X3 | | | |
| | | | | | | | @200-250 mg | | | |
| 3 | | | | | 130 | | | | | |
| 4 | | | | | 80 | | | | | |
| 5 | | | — | 0323256 | — | 10 | — | Q7D X3 | — | IV |
| | | | | Taxotere | | | | @200-250 mg | | |
| 6 | | | | | 5 | | | | | |
| 7 | | | | | 2.5 | | | | | |
| 8 | | | 0332991 | 0323256 | 210 | 10 | Q1Dx5,2D | 24 Hr post 5^{th} PO | PO | IV |
| | | | | Taxotere | | | off. X3 | dose X3 | | |
| | | | | | | | @200-250 mg | @200-250 mg | | |
| 9 | | | | | 130 | 10 | | | | |
| 10 | | | | | 80 | 10 | | | | |
| 11 | | | | | 210 | 5 | | | | |
| 12 | | | | | 130 | 5 | | | | |
| 13 | | | | | 80 | 5 | | | | |
| 14 | | | | | 210 | 2.5 | | | | |
| 15 | | | | | 130 | 2.5 | | | | |
| 16 | | | | | 80 | 2.5 | | | | |
| 17 | | | | | 210 | 10 | Q1Dx5, | Q7D X3 | PO | IV |
| | | | | | | | 24 H post | @200-250 mg | | |
| | | | | | | | IV X3 | | | |
| | | | | | | | @ 200-250 mg | | | |
| 18 | | | | | 130 | 10 | | | | |
| 19 | | | | | 80 | 10 | | | | |
| 20 | | | | | 210 | 5 | | | | |
| 21 | | | | | 130 | 5 | | | | |
| 22 | | | | | 80 | 5 | | | | |
| 23 | | | | | 210 | 2.5 | | | | |
| 24 | | | | | 130 | 2.5 | | | | |
| 25 | | | | | 80 | 2.5 | | | | |

Total No. Mice Required: 400 (192 extra)
Measure: 2 x's per week
Total Drug Required: see below FOR: Feb. 19, 2003
Weigh: To treat, then weekly × 4
PD0332991-0002B: 11.4 g parent needed
Taxotere, PD0323256: 96 mg parent

Chemotherapeutic Evaluation of a CDK-4 Inhibitor in Combination with the Standard Agent 5-Fluorouracil (5-FU) Against Advanced Stage Human Colon Carcinoma Colo-205

Objective: Determine the in vivo efficacy of the CDK-4 inhibitor 6-acetyl-8-cyclopentyl-5-methyl-2-(5-piperazin-1-yl-pyridin-2-ylamino)-8H-pyrido[2,3-d]pyrimidin-7-one and the standard agent 5-Fluorouracil (5-FU), when administered, as both single agents and together, in two combination schedules, against the solid human xenograft Colo-205.

Methods: Four hundred female SCID mice were received from Charles River Breeding Laboratories on Feb. 25, 2003. Mice were 21-28 days old upon arrival. All animals were examined prior to the initiation of the study to ensure that they were healthy and acclimated to the laboratory environment. Mice were housed in barrier facilities with food and water provided ad libitum on a 12-hour light/dark cycle. Animals housed 5 per cage while in the tumor pool, and 4 per cage when randomized into study groups. Animal care was provided in accordance with AAALAC guidelines. All protocols involving animals were reviewed and approved by the institutional animal care and use committee.

On day 0 (Mar. 13, 2003) all mice (18-22 g) received a subcutaneous (SC) implant of Colo-205 tumor fragment, approximately 30 mg, from tumors weighing 1000 mg. The solid human tumor model Colo-205, a colon carcinoma, was developed from cell lines and maintained in SCID mice. The tumor model was serially passed as SC implants of tumor fragments. Tumor source for this study was Colo-205/09B (T,2-11-03).

Treatment was initiated when tumor fragments reached 200-250 mg in size. Animals were randomized and then sorted into twenty-four groups of 8 mice. Animals were individually weighed and tumors measured the day treatment was initiated, and every 3-4 days during treatment. Dosing solutions were calculated according to average group weight. 6-Acetyl-8-cyclopentyl-5-methyl-2-(5-piperazin-1-yl-pyridin-2-ylamino)-8H-pyrido[2,3-d]pyrimidin-7-one and (5-FU) were prepared according to the drug preparation sheets, and as noted on the chemotherapy drug preparation data sheet. The compounds were administered as single agents, and in two different schedules. One set of animals was dosed with 6-acetyl-8-cyclopentyl-5-methyl-2-(5-piperazin-1-yl-pyridin-2-ylamino)-8H-pyrido[2,3-d]pyrimidin-7-one first for 5 days, followed in 24 hours by the standard agent (5-FU) for one IV dose. The second set was dosed with the standard agent (5-FU) for one dose, followed in 24 hours by 5 daily doses of 6-acetyl-8-cyclopentyl-5-methyl-2-(5-piperazin-1-yl-pyridin-2-ylamino)-8H-pyrido[2,3-d]pyrimidin-7-one. These schedules were designed to repeated three times, but due to the toxcitiy observed, only two dosing schedules were completed. The solution of 6-acetyl-8-cyclopentyl-5-methyl-2-(5-piperazin-1-yl-pyridin-2-ylamino)-8H-pyrido[2,3-d]pyrimidin-7-one was administered by oral gavage (PO) once daily, on days 20-24 and 27-31, or days 21-25, and 28-32, according to the treatment schedule. 5-FU was administered IV on days 25 and 32, or days 20 and 27, according to the treatment schedule. An additional group of 16 mice were included as negative control and were administered both dosing vehicles (50 mM sodium lactate for 6-acetyl-8-cyclopentyl-5-methyl-2-(5-piperazin-1-yl-pyridin-2-ylamino)-8H pyrido[2,3-d]pyrimidin-7-one, pH 4.0, and Saline for 5-FU) PO on days 20-24, and 27-31, and IV, on days 25 and 32. The PO dosing volume was 0.5 ml, the IV dosing volume was 0.2 ml.

During the course of the study the animals were weighed to calculate treatment doses and weekly thereafter. Tumors were measured every three to four days throughout the study. All animals were observed for clinical signs daily and after compound administration. All data collection was on CRAAS, and all printed reports were appropriately documented in the study file (databook 90663×12). This study was conducted in accordance with established departmental SOP's.

TABLE 2
CCTM Protocol Summary
ET- Expt. No.: 90663x12 Tumor: Col-205 Mouse: CB17 SCID Sex: F

| Cage | No. | | RX | | Dose (mg/kg/inj) | | Schedule | | Route | |
|---|---|---|---|---|---|---|---|---|---|---|
| No. | of Mice | Tumor Inoculum | Test | Standard | Test | Standard | Test | Standard | Test | Std. |
| 1 | 16 | +SC | Lactate | Saline | 0.5 mL | 0.2 mL | Q1Dx5,2D | 24 Hr post 5^{th} | PO | IV |
| | | | | | | | off. X3 | PO dose X3 | | |
| | | | | | | | @200-250 mg | @200-250 mg | | |
| 2 | | +SC | 0332991 | — | 210 | — | Q1Dx5,2D | — | PO | — |
| | | | | | | | off. X3 | | | |
| | | | | | | | @200-250 mg | | | |
| 3 | | | | | 130 | | | | | |
| 4 | | | | | 80 | | | | | |
| 5 | | | — | 0037760 | — | 100 | — | Q7D X3 | — | IV |
| | | | | 5FU | | | | @200-250 mg | | |
| 6 | | | | | | 62 | | | | |
| 7 | | | | | | 38 | | | | |
| 8 | | | 0332991 | 0037760 | 210 | 100 | Q1Dx5,2D | 24 Hr post 5^{th} | PO | IV |
| | | | | 5FU | | | off. X3 | PO dose X3 | | |
| | | | | | | | @200-250 mg | @200-250 mg | | |
| 9 | | | | | 130 | 100 | | | | |
| 10 | | | | | 80 | 100 | | | | |
| 11 | | | | | 210 | 62 | | | | |
| 12 | | | | | 130 | 62 | | | | |
| 13 | | | | | 80 | 62 | | | | |
| 14 | | | | | 210 | 38 | | | | |
| 15 | | | | | 130 | 38 | | | | |
| 16 | | | | | 80 | 38 | | | | |
| 17 | | | | | 210 | 100 | Q1Dx5, 24 H | Q7D X3 | PO | IV |
| | | | | | | | post IV X3 | @200-250 mg | | |
| | | | | | | | @200-250 mg | | | |
| 18 | | | | | 130 | 100 | | | | |
| 19 | | | | | 80 | 100 | | | | |
| 20 | | | | | 210 | 62 | | | | |
| 21 | | | | | 130 | 62 | | | | |
| 22 | | | | | 80 | 62 | | | | |
| 23 | | | | | 210 | 38 | | | | |
| 24 | | | | | 130 | 38 | | | | |
| 25 | | | | | 80 | 38 | | | | |

Total No. Mice Required: 400 (192 extra)
Measure: 2 x's per week
Total Drug Required: see below FOR: March 2003
weigh: To treat, then weekly × 4
PD0332991-0002B: 11.4 g parent needed
5FU, PD0037760 - mg parent

### Chemotherapeutic Evaluation of the CDK-4 Inhibitor 6-acetyl-8-cyclopentyl-5-methyl-2-(5-piperazin-1-yl-pyridin-2-ylamino)-8H-pyrido[2,3-d]pyrimidin-7-one in Combination with the Standard Agent Carboplatin, Against Advanced Stage Human Breast Carcinoma MDA-MB-435

Objective: Determine the in vivo efficacy of the CDK-4 inhibitor 6-acetyl-8-cyclopentyl-5-methyl-2-(5-piperazin-1-yl-pyridin-2-ylamino)-8H-pyrido[2,3-d]pyrimidin-7-one and the standard agent Carboplatin when administered, as both single agents and together, in two combination schedules, against the solid human xenograft MDA-MB-435.

Methods: Four hundred female SCID mice were received from Charles River Breeding Laboratories on Apr. 8, 2003. Mice were 21-28 days old upon arrival. All animals were examined prior to the initiation of the study to ensure that they were healthy and acclimated to the laboratory environment. Mice were housed in barrier facilities with food and water provided ad libitum on a 12-hour light/dark cycle. Animals housed 5 per cage while in the tumor pool, and 4 per cage when randomized into study groups. Animal care was provided in accordance with AAALAC guidelines. All protocols involving animals were reviewed and approved by the institutional animal care and use committee.

On day 0 (Apr. 24, 2003) all mice (18-22 g) received a subcutaneous (SC) implant of MDA-MB-435 tumor fragment, approximately 30 mg, from tumors weighing 1000 mg. The solid human tumor model MBA-MB-435, a breast carcinoma, was developed from cell lines and maintained in SCID mice. The tumor model was serially passed as SC implants of tumor fragments. Tumor source for this study was MDA-MB-435/17 (T, 4-2-03).

Treatment was initiated when tumor fragments reached 200-250 mg in size. Animals were randomized and then sorted into twenty-four groups of 8 mice. Animals were individually weighed and tumors measured the day treatment was initiated, and every 3-4 days during treatment. Dosing solutions were calculated according to average group weight. 6-Acetyl-8-cyclopentyl-5-methyl-2-(5-piperazin-1-yl-pyridin-2-ylamino)-8H-pyrido[2,3-d]pyrimidin-7-one and Carborplatin were prepared according to the drug preparation sheets, and as noted on the chemotherapy drug preparation data sheet. The compounds were administered as single agents, and in two different schedules. One set of animals was dosed with 6-acetyl-8-cyclopentyl-5-methyl-2-(5-piperazin-1-yl-pyridin-2-ylamino)-8H-pyrido[2,3-d]pyrimidin-7-one first for 5 days, followed in 24 hours by the standard agent (Carboplatin) for one IV dose. The second set was dosed with the standard agent (Carboplatin) for one dose, followed in 24 hours by 5 daily doses of 6-acetyl-8-cyclopentyl-5-methyl-2-(5-piperazin-1-yl-pyridin-2-ylamino)-8H-pyrido[2,3-d]pyrimidin-7-one. These schedules were repeated three times. The solution of 6-acetyl-8-cyclopentyl-5-methyl-2-(5-piperazin-1-yl-pyridin-2-ylamino)-8H-pyrido[2,3-d]pyrimidin-7-one was administered by oral gavage (PO) once daily, on days 14-18, 21-25, and 28-32, or days 15-19, 22-26, and 29-33, according to the treatment schedule. Carboplatin was administered IV on days 19, 26, and 33, or days 14, 21, and 28, according to the treatment schedule. An additional group of 16 mice were included as negative control and were administered both dosing vehicles (50 mM sodium lactate for 6-acetyl-8-cyclopentyl-5-methyl-2-(5-piperazin-1-yl-pyridin-2-ylamino)-8H-pyrido[2,3-d]pyrimidin-7-one, pH 4.0, and Saline for Carboplatin) PO on days 14-18, 21-25, and 28-32, and IV on days 19, 26, and 33. The PO dosing volume was 0.5 ml, the IV dosing volume was 0.2 ml.

During the course of the study the animals were weighed to calculate treatment doses and weekly thereafter. Tumors were measured every three to four days throughout the study. All animals were observed for clinical signs daily and after compound administration. All data collection was on CRAAS, and all printed reports were appropriately documented in the study file (databook 90663×15). This study was conducted in accordance with established departmental SOP's.

### Chemotherapeutic Evaluation of the CDK-4 Inhibitor 6-acetyl-8-cyclopentyl-5-methyl-2-(5-piperazin-1-yl-pyridin-2-ylamino)-8H-pyrido[2,3-d]pyrimidin-7-one and the MEK Inhibitor N-[(R)-2,3-Dihydroxy-propoxy]-3,4-difluoro-2-(2-fluoro-4-iodo-phenylamino)-benzamide and Against Advanced Stage Human Colon Carcinoma Colo-205

Objective: Determine the in vivo efficacy of the CDK-4/6 inhibitor 6-acetyl-8-cyclopentyl-5-methyl-2-(5-piperazin-1-yl-pyridin-2-ylamino)-8H-pyrido[2,3-d]pyrimidin-7-one and the MEK inhibitor N-[(R)-2,3-Dihydroxy-propoxy]-3,4-difluoro-2-(2-fluoro-4-iodo-phenylamino)-benzamide, when administered daily for 15 days, as both single agents and together, in combination, against the solid human xenograft Colo-205.

Methods: Four hundred female SCID mice were received from Charles River Breeding Laboratories on Jun. 17^{th}, 2003. Mice were 21-28 days old upon arrival. All animals were examined prior to the initiation of the study to ensure that they were healthy and acclimated to the laboratory environment. Mice were housed in barrier facilities with food and water provided ad libitum on a 12-hour light/dark cycle. Animals housed 5 per cage while in the tumor pool, and 4 per cage when randomized into study groups. Animal care was provided in accordance with AAALAC guidelines. All protocols involving animals were reviewed and approved by the institutional animal care and use committee.

On day 0 (Jul. 2, 2003) all mice (18-22 g) received a subcutaneous (SC) implant of Colo-205 tumor fragment, approximately 30 mg, from tumors weighing 1000 mg. The solid human tumor model Colo-205, a colon carcinoma, was developed from cell lines and maintained in SCID mice. The tumor model was serially passed as SC implants of tumor fragments. Tumor source for this study was Colo-205/13B (T,6-3-03).

Treatment was initiated when tumor fragments reached 200-250 mg in size. Animals were randomized and then sorted into twenty-four groups of 8 mice. Animals were individually weighed and tumors measured the day treatment was initiated, and every 3-4 days during treatment. Dosing solutions were calculated according to average group weight. 6-Acetyl-8-cyclopentyl-5-methyl-2-(5-piperazin-1-yl-pyridin-2-ylamino)-8H-pyrido[2,3-d]pyrimidin-7-one and N-[(R)-2,3-Dihydroxy-propoxy]-3,4-difluoro-2-(2-fluoro-4-iodo-phenylamino)-benzamide were prepared according to the drug preparation sheets, and as noted on the chemotherapy drug preparation data sheet. Solutions were administered by oral gavage (PO) once daily for 15 consecutive days. 6-Acetyl-8-cyclopentyl-5-methyl-2-(5-piperazin-1-yl-pyridin-2-ylamino)-8H-pyrido[2,3-d]pyrimidin-7-one was always given first, followed within one hour by N-[(R)-2,3-Dihydroxy-propoxy]-3,4-difluoro-2-(2-fluoro-4-iodo-phenylamino)-benzamide. An additional group of 16 mice was included as negative control and were administered both dosing vehicles (50 mM sodium lactate for 6-acetyl-8-cyclopentyl-5-methyl-2-(5-piperazin-1-yl-pyridin-2-ylamino)-8H-pyrido[2,3-d]pyrimidin-7-one, pH 4.0, and HPMT for N-[(R)-2,3-Dihydroxy-propoxy]-3,4-difluoro-2-(2-fluoro-4-iodo-phenylamino)-benzamide) PO, on the same daily treatment schedule. All dosing volumes were 0.5 ml.

During the course of the study the animals were weighed to calculate treatment doses and weekly thereafter. Tumors were measured every three to four days throughout the study. All animals were observed for clinical signs daily and after compound administration. All data collection was on CRAAS, and all printed reports were appropriately documented in the study file (databook 90665×15). This study was conducted in accordance with established departmental SOP's.

TABLE 3
CCTM Protocol Summary
MM- Expt. No.: 90665x015 Tumor: Colo-205 Mouse: CB17 SCID Sex: F

| Cage | No. | | RX | | Dose (mg/kg/inj) | | Schedule | | Route | |
|---|---|---|---|---|---|---|---|---|---|---|
| No. | of Mice | Tumor Inoc. | Test | Standard | Test | Standard | Test | Test#2 | Test | Test |
| 1 | 16 | +SC | Lactate | HPMT | 0.5 mL | 0.5 mL | QDx15 | QDx15 | PO | PO |
| 2 | 8 | +SC | 0332991 | — | 210 | — | QDx15 | | PO | |
| 3 | 8 | | | | 130 | | QDx15 | | PO | |
| 4 | 8 | | | | 80 | | QDx15 | | PO | |
| 5 | 8 | | | | 50 | | QDx15 | | PO | |
| 6 | 8 | | | 0325901 | | 25 | | QDx15 | | PO |
| 7 | 8 | | | | | 12 | | QDx15 | | PO |
| 8 | 8 | | | | | 6.25 | | QDx15 | | PO |
| 9 | 8 | | | | | 3.125 | | QDx15 | | PO |
| 10 | 8 | | 0332991 | 0325901 | 210 | 25 | QDx15 | QDx15 | PO | PO |
| 11 | 8 | | | | 130 | 25 | QDx15 | QDx15 | PO | PO |
| 12 | 8 | | | | 80 | 25 | QDx15 | QDx15 | PO | PO |
| 13 | 8 | | | | 50 | 25 | QDx15 | QDx15 | PO | PO |
| 14 | 8 | | | | 210 | 12 | QDx15 | QDx15 | P0 | PO |
| 15 | 8 | | | | 130 | 12 | QDx15 | QDx15 | PO | PO |
| 16 | 8 | | | | 80 | 12 | QDx15 | QDx15 | PO | PO |
| 17 | 8 | | | | 50 | 12 | QDx15 | QDx15 | PO | PO |
| 18 | 8 | | | | 210 | 6.25 | QDx15 | QDx15 | PO | PO |
| 19 | 8 | | | | 130 | 6.25 | QDx15 | QDx15 | PO | PO |
| 20 | 8 | | | | 80 | 6.25 | QDx15 | QDx15 | PO | PO |
| 21 | 8 | | | | 50 | 6.25 | QDx15 | QDx15 | PO | PO |
| 22 | 8 | | | | 210 | 3.125 | QDx15 | QDx15 | PO | P0 |
| 23 | 8 | | | | 130 | 3.125 | QDx15 | QDx15 | PO | PO |
| 24 | 8 | | | | 80 | 3.125 | QDx15 | QDx15 | PO | PO |
| 25 | 8 | | | | 50 | 3.125 | QDx15 | QDx15 | PO | PO |

Total No. Mice Required: 400 (192 extra)
Measure: 2 x's per week
Total Drug Required: see below FOR: Jul. 2, 2003
Weigh: To treat, then weekly × 4
PD0332991-0054: 11,000 mg parent needed- Rcvd. From CM, Lot Q, 78.2% P.
MEK Inhibitor: PD0325910-000: 1000 mg parent - in Lab, Lot S or T
NOTE:
Tumors will be staged to 200-250 mg.

### Chemotherapeutic Evaluation of the CDK-4 Inhibitor 6-acetyl-8-cyclopentyl-5-methyl-2-(5-piperazin-1-yl-pyridin-2-ylamino)-8H-pyrido[2,3-d]pyrimidin-7-one in Combination with the Standard Agent Camptosar (CPT-11), Against Advanced Stage Human Colon Carcinoma Colo-205

Objective: Determine the in vivo efficacy of the CDK-4 inhibitor 6-acetyl-8-cyclopentyl-5-methyl-2-(5-piperazin-1-yl-pyridin-2-ylamino)-8H-pyrido[2,3-d]pyrimidin-7-one and the standard agent Camptosar, Irinotecan (CPT-11), when administered, as both single agents and together, in two combination schedules, against the solid human xenograft Colo-205.

Methods: Three hundred female SCID mice were received from Charles River Breeding Laboratories on Jul. 8, 2003. Mice were 21-28 days old upon arrival. All animals were examined prior to the initiation of the study to ensure that they were healthy and acclimated to the laboratory environment. Mice were housed in barrier facilities with food and water provided ad libitum on a 12-hour light/dark cycle. Animals housed 5 per cage while in the tumor pool, and 4 per cage when randomized into study groups. Animal care was provided in accordance with AAALAC guidelines. All protocols involving animals were reviewed and approved by the institutional animal care and use committee.

On day 0 (Jul. 25, 2003) all mice (18-22 g) received a subcutaneous (SC) implant of Colo-205 tumor fragment, approximately 30 mg, from tumors weighing 1000 mg. The solid human tumor model Colo-205, a colon carcinoma, was developed from cell lines and maintained in SCID mice. The tumor model was serially passed as SC implants of tumor fragments. Tumor source for this study was Colo-205/14B (T, 7-2-03).

Treatment was initiated when tumor fragments reached 200-250 mg in size. Animals were randomized and then sorted into seventeen groups of 8 mice. Animals were individually weighed and tumors measured the day treatment was initiated, and every 3-4 days during treatment. Dosing solutions were calculated according to average group weight. 6-Acetyl-8-cyclopentyl-5-methyl-2-(5-piperazin-1-yl-pyridin-2-ylamino)-8H-pyrido[2,3-d]pyrimidin-7-one and CPT-11, were prepared according to the drug preparation sheets, and as noted on the chemotherapy drug preparation data sheet. The compounds were administered as single agents, and in two different schedules. One set of animals was dosed with 6-acetyl-8-cyclopentyl-5-methyl-2-(5-piperazin-1-yl-pyridin-2-ylamino)-8H-pyrido[2,3-d]pyrimidin-7-one first for 5 days, given two days without treatment, and then dosed for 5 additional days, followed in 2 days by the standard agent (CPT-11) for five daily IV doses. The second set was dosed with the standard agent (CPT-11) for five daily doses, followed in 2 days by 6-acetyl-8-cyclopentyl-5-methyl-2-(5-piperazin-1-yl-pyridin-2-ylamino)-8H-pyrido[2,3-d]pyrimidin-7-one dosed daily for 5 days, given two days without treatment, and then dosed for 5 additional days. The solution of 6-acetyl-8-cyclopentyl-5-methyl-2-(5-piperazin-1-yl-pyridin-2-ylamino)-8H-pyrido[2,3-d]pyrimidin-7-one was administered by oral gavage (PO) once daily, on days 17-21 and 24-28 or 24-28 and 31-35, according to the treatment schedule. CPT-11 was administered IV on days 17-21 or 31-35, according to the treatment schedule. An additional group of 16 mice were included as negative control and were administered both dosing vehicles (50 mM sodium lactate for 6-acetyl-8-cyclopentyl-5-methyl-2-(5-piperazin-1-yl-pyridin-2-ylamino)-8H-pyrido[2,3-d]pyrimidin-7-one, pH 4.0, and Saline for CPT-11) PO on days 17-21 and 24-28, and IV on days 31-35. The PO dosing volume was 0.5 ml, the IV dosing volume was 0.2 ml.

During the course of the study the animals were weighed to calculate treatment doses and weekly thereafter. Tumors were measured every three to four days throughout the study. All animals were observed for clinical signs daily and after compound administration. All data collection was on CRAAS, and all printed reports were appropriately documented in the study file (databook 90666×07). This study was conducted in accordance with established departmental SOP's.

TABLE 4
CCTM Protocol Summary
MM- Expt. No.: 90666x07 Tumor: Colo-205 Mouse: CB17 SCID Sex: F

| Cage | No. | | RX | | Dose (mg/kg/inj) | | Schedule | | Route | |
|---|---|---|---|---|---|---|---|---|---|---|
| No. | of Mice | Tumor Inoc. | Test | Standard | Test | Standard | Test | Stand. | Test | St. |
| 1 | 16 | +SC | Lactate | Saline | 0.5 mL | 0.2 mL | (QDx5)x2 then IV | 2D Post PO, | PO | IV |
| | | | | | | | | QDx5 | | |
| 2 | 8 | +SC | 0332991 | — | 210 | — | | | PO | |
| 3 | 8 | | | — | 130 | — | | | PO | |
| 4 | 8 | | | — | 80 | — | | | PO | |
| 5 | 8 | | | 0205014 | — | 50 | | QDx5 | | IV |
| 6 | 8 | | | | — | 25 | | QDx5 | | IV |
| 7 | 8 | | 0332991 | | 210 | 50 | (QDx5)x2 then IV | 2D post | PO | IV |
| | | | | | | | | PO, QDx5 | | |
| 8 | 8 | | | | 130 | 50 | | | PO | IV |
| 9 | 8 | | | | 80 | 50 | | | PO | IV |
| 10 | 8 | | | | 210 | 25 | | | PO | IV |
| 11 | 8 | | | | 130 | 25 | | | PO | IV |
| 12 | 8 | | | | 80 | 25 | | | PO | IV |
| 13 | 8 | | | | 210 | 50 | 2D post IV, | QDx5 | PO | IV |
| | | | | | | | (QDx5)x2 | | | |
| 14 | 8 | | | | 130 | 50 | | | PO | IV |
| 15 | 8 | | | | 80 | 50 | | | PO | IV |
| 16 | 8 | | | | 210 | 25 | | | PO | IV |
| 17 | 8 | | | | 130 | 25 | | | PO | IV |
| 18 | 8 | | | | 80 | 25 | | | PO | IV |

Total No. Mice Required: 300 (148 extra)
Measure: 2 x's per week
Total Drug Required: see below FOR: Jul. 25, 2003
Weigh: To treat, then weekly × 4
PD0332991: 6000 mg needed- UsePF-0080665-01, Lot 1 (80.42% P)-on shelf
Camptosar (CPT-11): PD0205014-000: 700 mg - in Lab Lot V
NOTE:
Tumors will be staged to 200-250 mg.

### Preparations

The following preparations are intended to be illustrative and non-limiting, and represent specific embodiments of the present invention.

### Preparation 1

### Preparation of 4-[6-(6-bromo-8-cyclopentyl-5-methyl-7-oxo-7,8-dihydro-pyrido[2,3-d]pyrimidin-2-ylamino)-pyridin-3-yl]-piperazine-1-carboxylic acid tert-butyl ester

A suspension of 6-bromo-8-cyclopentyl-2-methansulfinyl-5-methyl-8H-pyrido[2,3-d]pyrimidin-7-one (10.00 g, 0.027 mol, prepared as in Example 6 of WO 01/707041, which is incorporated herein by reference) and 10.37 g (0.0373 mol) of 4-(6-amino-pyridin-3-yl)-piperazine-1-carboxylic acid tert-butyl ester in toluene (100 mL) was heated under nitrogen in an oil bath for 7 hours. Thin layer chromatography (SiO₂, 10% MeOH/DCM) indicated the presence of both starting materials. The suspension was heated under reflux for an additional 18 hours. The resulting suspension was cooled to RT and filtered to give 4-[6-(6-bromo-8-cyclopentyl-5-methyl-7-oxo-7,8-dihydro-pyrido[2,3-d]pyrimidin-2-ylamino)-pyridin-3-yl]-piperazine-1-carboxylic acid tert-butyl ester (5.93 g, 38%). Melting point >250° C. MS (APCI) M⁺+1: calc'd, 584.2, found, 584.2.

### Preparation 2

### Preparation of 4-{6-[8-cyclopentyl-6-(1-ethoxy-vinyl)-5-methyl-7-oxo-7,8-dihydro-pyrido[2,3-d]pyrimidin-2-ylamino]-pyridin-3-yl}-piperazine-1-carboxylic acid tert-butyl ester

A suspension of 4-[6-(6-bromo-8-cyclopentyl-5-methyl-7-oxo-7,8-dihydro-pyrido[2,3-d]pyrimidin-2-ylamino)-pyridin-3-yl]-piperazine-1-carboxylic acid tert-butyl ester (5.93 g, 0.010 mol, prepared as in Example 1), tetrakis(triphenylphosphine)palladium(0) (1.40 g, 0.00121 mol), and tributyl(1-ethoxyvinyl)tin (5.32 mL, 0.0157 mol) in toluene (30 mL) was heated under reflux for 3.5 hours. The mixture was cooled and filtered to give a solid. Purification of the solid by silica gel chromatography using a gradient of 5%-66% ethyl acetate/hexane over 15 minutes gave 4-{6-[8-cyclopentyl-6-(1-ethoxy-vinyl)-5-methyl-7-oxo-7,8-dihydro-pyrido[2,3-d]pyrimidin-2-ylamino]-pyridin-3-yl}-piperazine-1-carboxylic acid tert-butyl ester as a yellow foam (4.50 g, 78%). MS (APCI) M⁺+1: calc'd 576.2, found, 576.3.

### Preparation 3

### Preparation of 6-acetyl-8-cyclopentyl-5-methyl-2-(5-piperazin-1-yl-pyridin-2-ylamino)-8H-pyrido[2,3-d]pyrimidin-7-one hydrochloride

Hydrogen chloride gas was bubbled into an ice-bath cooled solution of 4-{6-[8-cyclopentyl-6-(1-ethoxy-vinyl)-5-methyl-7-oxo-7,8-dihydro-pyrido[2,3-d]pyrimidin-2-ylamino]-pyridin-3-yl}-piperazine-1-carboxylic acid tert-butyl ester (4.50 g, 0.00783 mol, prepared as in Example 2) in DCM (100 mL). The resulting suspension was stoppered and stirred at RT overnight, then diluted with diethyl ether (200 mL). The solid was collected by filtration, washed with diethyl ether, and dried to give the hydrochloride salt of 6-acetyl-8-cyclopentyl-5-methyl-2-(5-piperazin-1-yl-pyridin-2-ylamino)-8H-pyrido[2,3-d]pyrimidin-7-one as a yellow solid (4.01 g, 92%). Melting point 200° C. HPLC, C18 reverse phase, 10%-95% gradient of 0.1% TFA/CH₃CN in 0.1% TFA/H₂O during 22 minutes: 99.0% at 11.04 minutes. MS (APCI) M⁺+1: calc'd, 448.2, found, 448.3. Anal. calc'd for C₂₄H₂₉N₇O₂.2.4H₂O.1.85 HCl: C, 51.64; H, 6.44; N, 17.56, Cl (total), 11.75. Found: C, 51.31; H, 6.41; N, 17.20; Cl (total), 12.11.

### Preparation 4

### Preparation of a mono-isethionate salt of 6-acetyl-8-cyclopentyl-5-methyl-2-(5-piperazin-1-yl-pyridin-2-ylamino)-8H-pyrido[2,3-d]pyrimidin-7-one (Form B)

To a slurry of 6-acetyl-8-cyclopentyl-5-methyl-2-(5-piperazin-1-yl-pyridin-2-ylamino)-8H-pyrido[2,3-d]pyrimidin-7-one (7.0 g, 15.64 mmol, prepared as in Example 3 following contact with NaOH) dispersed in 250 mL of water was added drop-wise 30 mL of a 0.52 M solution of isethionic acid in MeOH (15.64 mmol) to a pH of 5.2. The solution was filtered through a glass filter (fine) and the clear solution was freeze-dried to give 9.4 g of the amorphous salt. The amorphous salt (3.16 g) was mixed with 25 mL of MeOH and after almost complete dissolution a new precipitate formed. Another 25 mL of MeOH was added and the mixture was stirred at 46° C. to 49° C. for four hours. The mixture was slowly cooled to 32° C. and put in a cold room (+4° C.) overnight. A sample was taken for PXRD, which indicated formation of Form B. The mixture was filtered and the precipitate was dried overnight at 50° C. in a vacuum oven. This furnished 2.92 g of the mono-isethionate salt of the compound of Formula 1 in 92% yield. HPLC—99.25%, PXRD—Form B, CHNS, H-NMR were consistent with the structure.

### Preparation 5

### Preparation of a mono-isethionate salt of 6-acetyl-8-cyclopentyl-5-methyl-2-(5-piperazin-1-yl-pyridin-2-ylamino)-8H-pyrido[2,3-d]pyrimidin-7-one (Form B)

MeOH (100 mL) was placed in a 250 mL flask equipped with a mechanical stirrer, thermocouple/controller, condenser, and heating mantle and preheated to 35° C. An amorphous isethionate salt (2 g, prepared as in Example 4) was slowly added in three even portions with a 25 min to 30 min interval between the additions. The reaction mixture was stirred overnight at 35° C. and subsequently cooled. A sample was filtered and examined by PXRD. It was pure Form B. The whole reaction mixture was then used as Form B seeds in a larger scale experiment.

### Preparation 6

### Preparation of a mono-isethionate salt of 6-acetyl-8-cyclopentyl-5-methyl-2-(5-piperazin-1-yl-pyridin-2-ylamino)-8H-pyrido[2,3-d]pyrimidin-7-one (Form B)

MeOH (50 mL) was placed in a 250 mL flask equipped with a magnetic stirrer, condenser, thermocouple/controller, and heating mantle, and preheated to 40° C. An amorphous isethionate salt (1 g, prepared as in Example 4) was slowly added in three even portions with 30 min interval between the portions and then stirred overnight at 40° C. The reaction was monitored by in-situ Raman spectroscopy. The sample was taken, filtered and analyzed by PXRD. It was pure Form B by PXRD and Raman spectroscopy. The mixture was cooled to 25° C. at a rate of 3° C./h, cooled to −10° C., filtered, and vacuum dried to furnish 0.85 g of the Form B crystalline product.

### Preparation 7

### Preparation of a mono-isethionate salt of 6-acetyl-8-cyclopentyl-5-methyl-2-(5-piperazin-1-yl-pyridin-2-ylamino)-8H-pyrido[2,3-d]pyrimidin-7-one (Form B)

The free base (Formula 1, 0.895 mg, 2 mmol) was mixed with 10 mL of MeOH and seeded with 33 mg of a mono-isethionate salt of the compound of Formula 1 (Form B). Then 5.6 mL of a 0.375 M solution of isethionic acid in MeOH (2.1 mmol) was added in 10 even portions over 75 min time period. The mixture was stirred for an additional hour and a sample was taken for PXRD analysis. It confirmed formation of crystalline Form B. The mixture was stirred at RT overnight and another PXRD was taken. There was no change in the crystal form. The mixture was cooled in a refrigerator at −8° C. overnight, filtered, and dried at 50° C. in a vacuum oven to give 1.053 g (91.8% of theory) of the above-named compound (Form B). HPLC—99.8%, CHNS, H-NMR, IR are consistent with the structure, PXRD—Form B.

### Preparation 8

### Preparation of a mono-isethionate salt of 6-acetyl-8-cyclopentyl-5-methyl-2-(5-piperazin-1-yl-pyridin-2-ylamino)-8H-pyrido[2,3-d]pyrimidin-7-one (Form A)

An amorphous isethionate salt (47 mg, prepared as in Example 4) was mixed with 4 mL of EtOH in a 15 mL flask equipped with a magnetic stirrer, thermocouple and condenser. The mixture was heated to reflux, which resulted in the formation of a nearly clear solution. After refluxing for 10-15 min, the mixture became cloudy. It was slowly cooled to 50° C. and was seeded at 69° C. with Form A. The mixture was held at 50° C. for 5 h and was allowed to cool to RT overnight. The mixture was subsequently cooled to 1° C. with an ice bath, held for 1.5 h, filtered, washed with 0.5 mL of cold EtOH, air-dried, and then dried in a vacuum oven at 70° C. overnight to furnished 38.2 mg of a fine crystalline material. The crystalline material was found to be mono-isethionate salt Form A by PXRD. H-NMR was consistent for the mono-isethionate salt and indicated the presence of residual EtOH ca. 5.9 mol % or 0.6 wt %.

### Preparation 9

### Preparation of a mono-isethionate salt of 6-acetyl-8-cyclopentyl-5-methyl-2-(5-piperazin-1-yl-pyridin-2-ylamino)-8H-pyrido[2,3-d]pyrimidin-7-one (Form D)

An amorphous isethionate salt (9.0 g, prepared as in Example 4) was mixed with 300 mL of MeOH, stirred and heated to 63.8° C. (at reflux). To the slightly cloudy mixture was added two 50-mL portions of MeOH. The hot mixture was filtered into a 2-L flask equipped with a mechanical stirrer. The mixture was briefly heated to reflux and then cooled to 60° C. IPA (100 mL) was added to the mixture. The mixture was again heated to 60° C. and an additional 110 mL of IPA was added. A precipitate started to form at 59.7° C. The mixture was reheated to 67.5° C., cooled to 50° C., and held overnight. A sample was taken the next morning for PXRD analysis. The mixture was cooled to 25° C. at a rate of 3° C./h and another PXRD sample was taken when the mixture reached 28° C. The mixture was allowed to cool to RT overnight. A precipitate was collected and dried in a vacuum oven at 65° C. and 30 Torr. The procedure produced 7.45 g (82.8% yield) of the crystalline compound (Form D by PXRD analysis). Previously analyzed samples were also Form D. HPLC showed 98.82% purity and CHNS microanalysis was within +/−0.4%. A slurry of isethionate salt Form A, B, and D in MeOH yielded substantially pure Form B in less than three days.

### Preparation 10

### Preparation of isethionic acid (2-hydroxy-ethanesulfonic acid)

A 5-L, four-necked, round-bottomed flask, equipped with mechanical stirrer, thermocouple, gas sparger, and an atmosphere vent through a water trap was charged with 748 g (5.05 mol) of sodium isethionate (ALDRICH), and 4 L of IPA. The slurry was stirred at RT. An ice bath was used to keep the internal temperature below 50° C. as 925 g (25.4 mol) of hydrogen chloride gas (ALDRICH) was sparged into the system at a rate such that it dissolved as fast as it was added (as noted by lack of bubbling through the water trap). Sufficient HCl gas was added until the system was saturated (as noted by the start of bubbling through the water trap). During the addition of HCl, the temperature rose to 45° C. The slurry was cooled to RT and filtered over a coarse-fritted filter. The cake was washed with 100 mL of IPA and the cloudy filtrate was filtered through a 10-20μ filter. The resulting clear, colorless filtrate was concentrated under reduced pressure on a rotary evaporator, while keeping the bath temperature below 50° C. The resulting 1.07 kg of clear, light yellow oil was diluted with 50 mL of tap water and 400 mL of toluene and concentrated under reduced pressure on a rotary evaporator for three days, while keeping the bath temperature below 50° C. The resulting 800 g of clear, light yellow oil was diluted with 500 mL of toluene and 250 mL of IPA and concentrated under reduced pressure on a rotary evaporator for 11 days, keeping the bath temperature below 50° C. The resulting 713 g of clear, light yellow oil was titrated at 81 wt % (580 g, 91.1% yield) containing 7.9 wt % water and 7.5 wt % IPA.

### Preparation 11

### Preparation of 4-[6-[6-(1-butoxy-vinyl)-8-cyclopentyl-5-methyl-7-oxo-7,8-dihydro-pyrido[2,3-d]pyrimidin-2-ylamino]-pyridin-3-yl]-piperazine-1-carboxylic acid tert-butyl ester

A 5-L, three-necked, round-bottomed flask, equipped with a mechanical stirrer, a thermocouple, and a nitrogen inlet/outlet vented through a silicone oil bubbler was placed under a nitrogen atmosphere and charged with 4-[6-(6-bromo-8-cyclopentyl-5-methyl-7-oxo-7,8-dihydro-pyrido[2,3-d]pyrimidin-2-ylamino)-pyridin-3-yl]-piperazine-1-carboxylic acid tert-butyl ester (300 g, 0.51 mol, prepared as in Example 2), butyl vinyl ether (154 g, 1.54 mol, ALDRICH), n-butanol (1.5 L, ALDRICH), and diisopropyl ethylamine (107 mL, 0.62 mol, ALDRICH). The slurry was placed under approximately 50 Torr vacuum and then refilled with nitrogen 3 times. To this was added 8.3 g (0.01 mol) bis-(diphenylphosphinoferrocene) palladium dichloride dichloromethane (Johnson Matthey, Lot 077598001) and the resulting slurry was purged an additional three times as described above. The mixture was then heated to 95° C. and stirred for 20 h. The resulting thin red slurry was diluted with 2 L of heptane and cooled to approximately 5° C. At this temperature, 400 mL saturated aqueous potassium carbonate was added and the mixture was filtered and rinsed with 250 mL of heptane. After drying in an oven for 16 h at 45° C., 231.7 g (75% yield) of the title compound was obtained as a yellow solid.

### Preparation 12

### Preparation of a mono-isethionate salt of 6-acetyl-8-cyclopentyl-5-methyl-2-(5-piperazin-1-yl-pyridin-2-ylamino)-8H-pyrido[2,3-d]pyrimidin-7-one (Form B)

A 22-L, three-necked, round-bottomed flask, equipped with a mechanical stirrer, a thermocouple, and a nitrogen inlet/outlet vented through a silicone oil bubbler was placed under a nitrogen atmosphere and charged with 4-{6-[6-(1-butoxy-vinyl)-8-cyclopentyl-5-methyl-7-oxo-7,8-dihydro-pyrido[2,3-d]pyrimidin-2-ylamino]-pyridin-3-yl}-piperazine-1-carboxylic acid tert-butyl ester (725 g, 1.20 mol, prepared as in Example 11) and MeOH (14 L). The slurry was stirred at RT as it was charged with a solution of isethionic acid (530 g, 4.20 mol, prepared as in Example 10), MeOH (1.5 L), and water (70 mL, 3.89 mol). The resulting slurry was heated to 55° C. over 30 minutes and then stirred at 55° C. for 30 minutes. A solution of 175 g (1.73 mol) of Et₃N (ALDRICH) in 200 mL of MeOH was charged to the slurry as it was cooled to 30° C. The slurry was held at 30° C. as a solution of 128 g (1.26 mol) of Et₃N in 2 L of MeOH was added dropwise over 6 hours. The resulting slurry was sampled to determine crystal form (Form B). The slurry was cooled and held at 5° C. for 15 minutes and was subsequently filtered through a coarse-fritted filter. The resulting filter cake was washed with multiple washes of 200 mL of cold MeOH. The solid product was dried at 55° C. under vacuum to yield 710 g (91% yield) of the title compound as yellow crystals.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patents, patent applications, and patent publications, are incorporated herein by reference in their entirety and for all purposes.

### Preparation 13

### N-[(R)-2,3-Dihydroxy-propoxy]-3,4-difluoro-2-(2-fluoro-4-iodo-phenylamino)-benzamide (Form IV)

To a flask containing 3,4-difluoro-2-(2-fluoro-4-iodo-phenylamino)-benzoic acid (2.6 Kg, 6.6 mol) and N,N′-carbonyldiimidazole (1.1 Kg, 6.8 mol) under nitrogen atmosphere, was added 12 L of dry acetonitrile. After stirring at 220±5° C. for about 90 minutes, a solution of (R)-O-(2,2-dimethyl-[1,3]dioxolan-4-ylmethyl)-hydroxylamine in toluene was added (8.5 L total volume, about 8 moles of amine). The solution was stirred for at least 6 hours at 22°±5 C. Aqueous hydrochloric acid (9 L, 1.5 molar) was added, and after stirring for about 5 minutes, the layers were separated. Aqueous hydrochloric acid (9 L, 1.5 molar) was added to the remaining top layer, and after stirring for about 20 hours, the layers were separated. The remaining top layer was concentrated by vacuum distillation, and then diluted with 15 L toluene and 2 L ethanol. The mixture was warmed to 35-45° C. and diluted with 20 L warm water, then cooled to 0-5° C. The product was collected by filtration and washed with 2 L toluene. The product was recrystallized by dissolving in 12 L toluene and 2 L ethanol (50°±5 C), adding 10 L water and cooling to 0-5° C. After collecting the product by filtration and washing with toluene, the product was dried in a vacuum oven resulting in 2.6 Kg of N-[(R)-2,3-Dihydroxypropoxy]-3,4-difluoro-2-(2-fluoro-4-iodo-phenylamino)-benzamide.

2.4 Kg of the above compound as a mixture of different crystalline forms was stirred in a mixture of 10 L water and 1 L ethanol at 35±5° C. for 20-30 hours, then cooled to 25±5 C. The product was collected by filtration and washed with 1 L of water, then dried in a vacuum oven at 65° C. This resulted in 2.3 Kg of material which was greater than 90% form IV. Note: DSC analysis shows an onset of melting at 110° C. with only a small amount of the peak with an onset of melting at 117° C.

1. A method for treating abnormal cell growth in a patient in need of such treatment, the method comprising administering to the patient a combination of an amount of a selective CDK inhibitor and an amount of one or more signal transduction inhibitors, wherein the amounts of the selective CDK inhibitor and the signal transduction inhibitor(s) when taken as a whole is therapeutically effective for treating said abnormal cell growth.

2. The method according to claim 1, the method comprising administering to the patient a combination of an amount of a selective CDK-4 inhibitor, CDK-6 inhibitor or CDK-4/6 inhibitor and an amount of one or more signal transduction inhibitors, wherein the amounts of the selective CDK-4 inhibitor, CDK-6 inhibitor or CDK-4/6 inhibitor and the signal transduction inhibitor(s) when taken as a whole is therapeutically effective for treating said abnormal cell growth.

3. The method according to claim 2, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of one or more signal transduction inhibitors, wherein the amounts of the selective CDK4/6 inhibitor and the signal transduction inhibitor(s) when taken as a whole is therapeutically effective for treating said abnormal cell growth.

4. The method according to claim 3, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of a MEK inhibitor, wherein the amounts of the CDK4/6 inhibitor and the MEK inhibitor when taken as a whole is therapeutically effective for treating said abnormal cell growth.

5. The method according to claim 4, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of the MEK inhibitor 2-(2-chloro-4-iodo-phenylamino)-N-cyclopropylmethoxy-3,4-difluoro-benzamide, wherein the amounts of the CDK4/6 inhibitor and the MEK inhibitor when taken as a whole is therapeutically effective for treating said abnormal cell growth.

6. The method according to claim 4, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of the MEK inhibitor N-[(R)-2,3-Dihydroxy-propoxy]-3,4-difluoro-2-(2-fluoro-4-iodo-phenylamino)-benzamide, wherein the amounts of the CDK4/6 inhibitor and the MEK inhibitor when taken as a whole is therapeutically effective for treating said abnormal cell growth.

7. The method according to claim 3, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of a signal transduction inhibitor selected from the group consisting of Raf kinase inhibitor, Akt inhibitor and mTOR inhibitor, wherein the amounts of the CDK4/6 inhibitor and the signal transduction inhibitors when taken as a whole is therapeutically effective for treating said abnormal cell growth.

8. The method according to claim 7, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of a Raf kinase or mTOR inhibitor selected from the group consisting of BAY 43-9006, rapamycin, CCI 779, Rad001 or Arry 142886, wherein the amounts of the CDK4/6 inhibitor and the Raf kinase or mTOR inhibitor when taken as a whole is therapeutically effective for treating said abnormal cell growth.

9. The method according to claim 3, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of one or more signal transduction inhibitors selected from the group consisting of bcr-abl tyrosine kinase inhibitors, PDGFR inhibitors, c-Kit inhibitors, erbB inhibitors, VEGF-R inhibitors, FGFR inhibitors and IGF1-R inhibitors, wherein the amounts of the CDK4/6 inhibitor and the signal transduction inhibitor(s) when taken as a whole is therapeutically effective for treating said abnormal cell growth.

10. The method according to claim 9, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of a PDGFR, erbB, or VEGF-R inhibitor selected from the group consisting of CP-868,596, ST-1571, PTK-787, PKC-412, Herceptin (trastuzumab), Erbitux, Iressa (gefitinib), Tarceva (erlotinib), EKB-569, PKI-166, GW-572016, E-2-methoxy-N-(3-{4-[3-methyl-4-(6-methyl-pyridin-3-yloxy)-phenylamino]-quinazolin-6-yl}-allyl)-acetamide, CI-1033, CP-547,632, PTK 787, ZD 6474, PKC 412 and Avastin (Bevacizumeb), wherein the amounts of the CDK4/6 inhibitor and the PDGFR, erbB or VEGF-R inhibitor when taken as a whole is therapeutically effective for treating said abnormal cell growth.

11. A method for treating abnormal cell growth in a patient in need of such treatment, the method comprising administering to the patient a combination of an amount of the CDK4/6 inhibitor 6-acetyl-8-cyclopentyl-5-methyl-2-(5-piperazin-1-yl-pyridin-2-ylamino)-8H-pyrido[2,3-d]pyrimidin-7-one and an amount of a muliti-targeted kinase inhibitor, wherein the amounts of the CDK4/6 inhibitor and the multi-targeted inhibitor when taken as a whole is therapeutically effective for treating said abnormal cell growth.

12. The method according to claim 11, the method comprising administering to the patient a combination of an amount of the CDK inhibitor 6-acetyl-8-cyclopentyl-5-methyl-2-(5-piperazin-1-yl-pyridin-2-ylamino)-8H-pyrido[2,3-d]pyrimidin-7-one and an amount of a multi-targeted kinase inhibitor is SU11248 or Gleevec, wherein the amounts of the CDK4/6 inhibitor and the multi-targeted inhibitor when taken as a whole is therapeutically effective for treating said abnormal cell growth.

13. The method according to claim 1, the method further comprising administering to the patient one or more additional therapeutic agents selected from the group consisting of an antitumor agent, alkylating agent, antimetabolite, antibiotic, plant-derived antitumor agent, camptothecin derivative, interferon, and biological response modifier.

14. The method according to claim 5, the method further comprising administering to the patient one or more additional therapeutic agents selected from the group consisting of an antitumor agent, alkylating agent, antimetabolite, antibiotic, plant-derived antitumor agent, camptothecin derivative, interferon, and biological response modifier.

15. The method according to claim 11, the method further comprising administering to the patient one or more additional therapeutic agents selected from the group consisting of an antitumor agent, alkylating agent, antimetabolite, antibiotic, plant-derived antitumor agent, camptothecin derivative, interferon, and biological response modifier.

16. The method according to claim 1, the method further comprising administering to the patient one or more additional therapeutic agents selected from the group consisting of cis-platin, oxaliplatin, carboplatin, cyclophosphamide, 5-fluorouracil, capecitabine, cytosine arabinosid, hydroxyurea, N-(5-[N-(3,4-dihydro-2-methyl-4-oxoquinazolin-6-ylmethyl)-N-methylamino]-2-thenoyl)-L-glutamic acid, adriamycin, bleomycin, interferon, Nolvadex (tamoxifen), and Casodex (4′-cyano-3-(4-fluorophenylsulphonyl)-2-hydroxy-2-methyl-3′-(trifluoromethyl)propionanilide).

17. The method according to claim 5, the method further comprising administering to the patient one or more additional therapeutic agents selected from the group consisting of cis-platin, oxaliplatin, carboplatin, cyclophosphamide, 5-fluorouracil, capecitabine, cytosine arabinosid, hydroxyurea, N-(5-[N-(3,4-dihydro-2-methyl-4-oxoquinazolin-6-ylmethyl)-N-methylamino]-2-thenoyl)-L-glutamic acid, adriamycin, bleomycin, interferon, Nolvadex (tamoxifen), and Casodex (4′-cyano-3-(4-fluorophenylsulphonyl)-2-hydroxy-2-methyl-3′-(trifluoromethyl)propionanilide).

18. The method according to claim 11, the method further comprising administering to the patient one or more additional therapeutic agents selected from the group consisting of cis-platin, oxaliplatin, carboplatin, cyclophosphamide, 5-fluorouracil, capecitabine, cytosine arabinosid, hydroxyurea, N-(5-[N-(3,4-dihydro-2-methyl-4-oxoquinazolin-6-ylmethyl)-N-methylamino]-2-thenoyl)-L-glutamic acid, adriamycin, bleomycin, interferon, Nolvadex (tamoxifen), and Casodex (4′-cyano-3-(4-fluorophenylsulphonyl)-2-hydroxy-2-methyl-3′-(trifluoromethyl)propionanilide).

19. A pharmaceutical combination comprising an amount of 6-acetyl-8-cyclopentyl-5-methyl-2-(5-piperazin-1-yl-pyridin-2-ylamino)-81+pyrido[2,3-d]pyrimidin-7-one or an isethionate salt thereof and an amount of one or more signal transduction inhibitors selected from the group consisting of tyrosine kinase inhibitors, MEK inhibitors, bcr-abl tyrosine kinase inhibitors, PDGFR inhibitors, c-Kit inhibitors, erbB inhibitors, VEGF-R inhibitors, Hsp 90 inhibitors, Aurora kinase inhibitos, FLT-3 inhibitors, n-Ras inhibitors, PI3 kinase inhibitors, Raf kinase inhibitors, Akt inhibitors, mTOR inhibitors and multitargeted kinase inhibitors.
